(19) United States
(12) Patent Application Publication
(10) Pub. No.: US 2019/0242883 A1
(43) Pub. Date: Aug. 8, 2019

(54) PROTEIN-COATED MICROPARTICLES FOR PROTEIN STANDARDIZATION IN SINGLE-CELL ASSAYS

(71) Applicant: The Regents of the University of California, Oakland, CA (US)

(72) Inventors: Amy E. Herr, Berkeley, CA (US); Pui Yik Chan, Berkeley, CA (US)

(21) Appl. No.: 16/271,483

(22) Filed: Feb. 8, 2019

Related U.S. Application Data
(60) Provisional application No. 62/628,270, filed on Feb. 8, 2018.

(51) Int. Cl. G01N 33/533; B01L 3/00; G01N 15/14; G01N 27/447; G01N 33/543; G01N 33/544

(52) U.S. Cl. CPC G01N 33/533; B01L 3/502715; G01N 15/1484; B01L 3/502761; G01N 27/44791; G01N 2021/1746; G01N 33/54326; G01N 33/544; G01N 33/54353; G01N 2015/1481; G01N 33/5432

(57) ABSTRACT

The disclosure provides for protein-coated micro- or nano-particles that can be used as reference standards for assessing technical variations in microfluidic devices.

### CROSS REFERENCE TO RELATED APPLICATIONS

This application claims priority under 35 U.S.C. § 119 from Provisional Application Ser. No. 62/628,270, filed Feb. 8, 2018, the disclosures of which are incorporated herein by reference.

### TECHNICAL FIELD

The disclosure provides for protein-coated microparticles that can be used as reference standards for assessing technical variations in microfluidic devices.

### BACKGROUND

Microfluidics has enabled high-throughput protein measurement from thousands of individual cells and holds great promise for precision medicine. A lack of standardization, however, has suppressed implementation of single-cell protein tools in quantitative medicine.

### SUMMARY

Provided herein are microparticles that can be used as reference standards for assessing technical variations in microfluidic devices (e.g., single-cell electrophoretic cytometry). The microparticles disclosed herein can be functionalized with click chemistry to reversibly append or reversibly attach tracer biomolecules to the microparticles. Moreover, the microparticles of the disclosure can be implemented within a microdevice by using magnetic fields, using passive-gravity, and/or by using centrifugal methods. The release of tracer biomolecules from the microparticles can readily be measured. Thus, the microparticles of the disclosure can be used as standards for assessing technical variation and noise within the microfluidic devices. The release of the tracer biomolecules from the microparticles disclosed herein can be brought about by the introduction of a releasing reagent (e.g., competitive ligand) into the microfluidic device buffer. The released tracer biomolecules can be measured optically in situ.

In a particular embodiment, the disclosure provides for a method for controlling biomolecule measurement quality in a microfluidic device, comprising: introducing into a microfluidic device one or more nitrilotriacetic acid (NTA)-functionalized microparticles that comprise one or more reversibly attached tracer biomolecules; releasing the tracer biomolecules from the microparticles by using a releasing reagent in a buffer and/or heating; and measuring the released tracer biomolecules. In a further embodiment, the NTA-functionalized particles comprising one or more reversibly attached tracer biomolecules are introduced into the microfluidic device by either using passive gravity, magnetic attraction, or centrifugal forces. In a certain embodiment, the NTA-functionalized particles are NTA-functionalized magnetic particles. In a further embodiment, the NTA-functionalized magnetic particles comprise Fe₂O₃ or Fe₃O₄. In an alternate embodiment, the NTA-functionalized particles are NTA-functionalized polystyrene, silica, or polyketal particles. In another embodiment, the one or more tracer biomolecules are reversibly attached to the NTA-functionalized particles using chelation click chemistry. Examples of one or more tracer biomolecules include, but are not limited to, proteins, peptides or ribosomes. In yet a further embodiment, the one or more tracer biomolecules are one or more proteins that differ by molecular weight. In a particular embodiment, the one or more proteins are fluorescent proteins. In a further embodiment, the one or more tracer biomolecules comprise polyhistidine tags. In another embodiment, the one or more proteins comprise 6× histidine tags located at the C′ or N′ terminus of the proteins. In yet another embodiment, the releasing agent is a competitive ligand. In a certain embodiment, the releasing agent is imidazole. In a further embodiment, the released tracer biomolecules can be measured by measuring fluorescent light intensity. In yet a further embodiment, the released tracer biomolecules can be measured by using an antibody that is linked to a reporter enzyme that is capable of cleaving chemiluminescent agents, and measuring luminescent light intensity. In another embodiment, the microfluidic device is a single-cell mass cytometry device.

In a particular embodiment, the disclosure further provides for a method for using protein-coated microparticles for measurement standardization in single-cell protein assays, comprising: introducing into one or more wells of a single cell electrophoretic cytometry device: (i) a cell; and (ii) one or more nitrilotriacetic acid (NTA)-functionalized microparticles that comprise one or more reversibly attached proteins; releasing the one or more proteins from the microparticles by using a cell lysis buffer which comprises imidazole; and optionally performing buffer exchange to remove the imidazole; separating the one or more proteins by applying an electric field; immobilizing the one or more proteins by using ultra violet light; quantifying the one or more proteins using immunoprobing or by measuring fluorescence or luminescence light intensity. In a further embodiment, the single-cell protein assay is single cell-resolution western blotting. In yet a further embodiment, the cell and the one or more NTA-functionalized microparticles are loaded into wells of a polyacrylamide gel electrophoresis (PAGE) gel. In another embodiment, the one or more proteins comprise polyhistidine tags and are reversibly attached to the NTA-functionalized particles using chelation click chemistry.

### DESCRIPTION OF DRAWINGS

FIG. 1A-C provides various diagrams and schemes showing the reversible functionalization of the microparticles of the disclosure with proteins and the use of the microparticles for electrophoretic cytometry protein sizing. (A) Diagram illustrating protein release from nitrilotriacetic acid (NTA) coated microparticles. (B) Diagram illustrating workflow using the microparticles in delivering protein standards in single cell-resolution western blotting (scWB). (C) Representative photo of scWB gel and fluorescence images showing a gel resolving protein ladder in a gel comprising an array of micro-wells.

FIG. 2A-B presents the effects of temperature and imidazole concentration on protein release kinetics. (A) Representative fluorescence micrographs showing protein release from microparticles under 4° C., room temperature (R.T.), and 50° C. lysis condition. Scale bar represents 30 μm length. (B) Time course of the fluorescence intensity (represented as relative fluorescence units (RFUs)) from the microwell of (A).

FIG. 3A-B shows that proteins released from the microparticles are electrophoretically separated yielding broad molecular weight-range protein ladders for thousands of simultaneous single-cell electrophoretic separations. (A) Representative false-color micrograph montage and y-offset intensity profiles (maximum normalized) of a time-lapse image of electrophoretic separations of Protein A (34 kDa) and Protein A/G (50.4 kDa) released from the microparticles. (B) Representative false-color micrographs and intensity profiles of a four-protein ladder that was separated and immobilized. Inset shows the microwell outlined with black and microparticles remaining in the well. Mean separation resolution (Rₛ) for each protein pair is shown on the intensity profile (error bars are standard deviation for n=322 separations). Scale bars are 100 microns.

FIG. 4A-B provides for the quantitative determinization of the number of microbeads required per microwell for detectable protein peak signal. (A) Representative false-color micrographs and intensity profiles (from median filtered images) of a four-protein ladder. (B) Quantitation of ICAM1 peak signal to noise ratio (SNR) (from median-filtered images) as a function of the number of microbeads in the microwell (n=4 microbeads, error bar is standard deviation). At least 4 microbeads are required per microwell to achieve a SNR>3.

FIG. 5A-B shows buffer exchange from 1M imidazole to 0M imidazole buffers restores immunoprobing signals. (A) Diagrams illustrating chemical lysis and electrophoresis with or without buffer exchange. (B) Box-and-whisker plots of 3-TUB, CK8, GAPDH distributions under different buffer conditions are measured by an area-under-the-curve analysis (Krusakal Wallis test, Dunn-Šidák corrections, *<0.05, **<0.0001).

FIG. 6A-C shows that ladder proteins from microparticles migrate according to molecular weight and that the molecular weights of the protein targets from single cells were confirmed. (A) A top-left false-color micrograph displays a microwell containing a MCF-7 cell and two microparticles. Scale bar, 5 μm. Right false-color micrographs present scWB results of 4 representative lanes containing both protein targets (STAT3, 80 kDA; ER-α, 66 kDA; CK8, 54 kDA; β-TUB, 50 kDA; GAPDH, 37 kDA) and ladder proteins (blue). (B) False-color micrographs, intensity profiles, and Ferguson plots (log MW vs separation distance) of each target protein. (C) A box-and-whicker plot depicts calculated molecular weights of STAT3, ER-α, CK8, β-TUB, GAPDH molecular weights from Ferguson plots.

FIG. 7 presents a diagram indicating how there is a need for technical variation assessment in electrophoresis to measure cell-to-cell variations.

FIG. 8 presents a diagram and equations demonstrating that the microparticles of the disclosure can be standardized vehicles for protein loading and release.

FIG. 9 presents a diagram showing how the microparticles can as be used as vehicles for protein standard loading and release.

FIG. 10 presents a diagram and graph showing the importance of buffer exchange for removing imidazole interference in antibody probing.

FIG. 11 presents pictures, figures and graphs demonstrating the validation and assessment of protein mass sizing using the microparticles of the disclosure.

FIG. 12A-E shows a further schematic and resulting data of the disclosure. (A) Schematic of the single-cell western blot workflow that incorporates microparticles to deliver protein markers for molecular mass determination of endogenous proteins from mammalian cells. The single-cell western blot comprises, in certain embodiments, five stages: 1) Cell settling into microwells by gravity (F_{g,z}), 2) Microparticle settling into microwells by an applied magnetic field (z-axis) (B_{z}), 3) In-microwell protein marker release (imidazole) and chemical cell lysis, 4) Concurrent polyacrylamide gel electrophoresis and photo-activated immobilization (photoblotting) of protein markers and single-cell lysate, and 5) In-gel immunoprobing. (B) Microwell occupancy of MCF-7 cells and protein-coated microparticles. False-colour micrographs of microwells housing a cell and/or microparticles. Scale bar is 15 μm. Distributions of microwell occupancy for cells and microparticles. Error bars are standard deviations (n=3 chips). (C) Schematic of His-tagged protein marker delivery and release from Ni²⁺-coated microparticles. (D) Time-lapse fluorescence signal during 1 M imidazole-trigged release of a fluorescent protein marker from a single protein-loaded microparticle in a microwell (n=3 microwells, error bars are standard deviation). Dashed lines represent exponential curve fitting. The marker release half-life for each condition is listed. Black (bottom): y=1.04*exp(−0.16t)+0.11, R²=0.91. Red (middle): y=1.18*exp(−0.04t)+0.08, R²=0.90; Blue (top): y=1.03*exp(−0.01t)−0.09, R²=0.87. (E) False-colour micrograph and intensity profile of four standard proteins (ICAM1, 100 kDa; KDR, 85 kDa; EpH B4, 58 kDa; CHI3L1, 42 kDa) after electrophoresis separation and photoblotting in the polyacrylamide gel. The 30-μm diameter microwell is at left of micrograph.

FIG. 13A-C shows a Ferguson analyses of protein markers support utility as an estimator of molecular mass for endogenous protein targets. (A) Log-linear regression fitting of protein markers (ICAM1, KDR, EpH B4, CHI3L1) in 100 representative single-cell PAGE protein separations. R² value for each linear fit is shown in lower left of each plot. Black circles, protein markers; lines, linear regression fits. (B) Box and scatter plots of percent mass errors between expected and estimated (from the log-linear regression fits) molecular masses of protein markers in a single-cell western blot chip. Each black circle represents percent mass error for each PAGE separation lane. Box extents indicate 25th and 75th quantiles; black line at box midpoint indicates median value; whiskers extend to minimum and maximum values. Kruskal-Wallis test with Dunn's multiple comparisons test, ***p<0.0001, n=168 PAGE separation lanes. (C) Scatter plots indicate chip-to-chip R² values for linear regression fits. Black circles indicate R² values for PAGE separation lanes; red line indicates mean value for each chip (μ₁=0.991, μ₂=0.994, μ₃=0.993); n₁=327, n₂=204, n₃=516; one-way ANOVA test, p<0.001, Cohen's d<0.2.

FIG. 14A-F shows imidazole interferes with protein blotting efficiency thus necessitating buffer exchange prior to UV exposure. (A) False-colour micrographs of single-cell protein PAGE with 1 M imidazole present during photoblotting (left) and with buffer exchange to dilute imidazole prior to photoblotting (right). Each intensity plot represents the protein markers along one PAGE separation lane. (B) Scatter plots of signal-to-noise ratio (SNR) of protein marker peaks with and without buffer exchange (n_{with}_{_}_{exchange}=249, nₙₒ_{_}_{exchange}=149). (C) Scatter plot indicating R² values with and without buffer exchange. (n_{with}_{_}_{exchange}=107, nₙₒ_{_}_{exchange}=27). (D) Scatter plots of separation resolution between two protein markers with or without buffer exchange (n_{with}_{_}_{exchange}=107, nₙₒ_{_}_{exchange}=27). (E) Scatter plot of KDR peak SNR as a function of the number of microparticles in the microwell (n=4 microwells, error bar is standard deviation). Each square represents a mean SNR value of KDR with buffer exchange. Each circle represents a mean SNR value of KDR without buffer exchange. With buffer exchange, one microparticle per microwell is sufficient to detect all protein markers with SNR>3. (F) Bar graph representing fraction of protein markers that passed quality control (SNR>3, R² for Gaussian curve fitting≥0.7) Error bars represent standard deviations, n=3 chips with exchange and without exchange. Black circles represent data per PAGE separation lane; red lines represent mean values; unpaired t test, *** p<0.0001, ** p<0.002, ns=no significance (p>0.05).

FIG. 15A-F shows the ability of protein markers to estimate protein target molecular mass in single-cell western blotting. (A) false-colour micrographs display microwells containing an MCF-7 cell and microparticles. Scale bar, 10 μm; intensity profiles and false-colour micrographs of four representative PAGE lanes containing both endogenous proteins (STAT3, 80 kDa; ER-α, 66 kDa; CK8, 54 kDa; β-TUB, 50 kDa; GAPDH, 37 kDa) and protein markers. (B) Scatter plots indicate no significant difference in peak widths of protein markers with and without cells in microwells. Black circles represent peak width of each protein marker; lines represent mean values; unpaired t test and F test, ns=no significance (p>0.05); n_{with}_{_}_{cell}=141, nₙₒ_{_}_{cell}=127. (C) Scatter plot indicates no significant difference in R² values from linear regression with and without a cell in the microwell. Black circles indicate R² values per separation lane; lines represent mean values; unpaired t test and F test, ns=no significance (p>0.05); n_{with}_{_}_{cell}=141, nₙₒ_{_}_{cell}=127 (D) Scatter plot representing no significance difference in GAPDH (n_{≥1}=55, n₀=17), β-TUB (n_{≥1}=77, n₀=28), and CK8 (n_{≥1}=97, n₀=14) peak widths in microwells with or without microparticles. Black circles indicate endogenous protein peak width per separation lane; lines represent mean values; Mann-Whitney test, ns=no significance (p>0.05). (E) Representative log-linear regression plots display estimated (*) and expected (x) molecular masses of endogenous proteins extracted from linear regression of the protein marker. R² value for each log-linear fit is in the lower left of each plot. Black circles, protein marker; lines, log-linear regression fitting. (F) Box plots depict % error between estimated and expected molecular masses of STAT3 (n=95 cells), ER-α (n=7 cells), CK8 (n=794 cells), β-TUB (n=551 cells), and GAPDH (n=430 cells) from the Ferguson analysis plots. Box extents indicate 25th and 75th quantiles; black line at box midpoint indicates median value; whiskers extend to minimum and maximum values.

FIG. 16 shows scatter plots of β-TUB, CK8, GAPDH expressions under different lysis buffer conditions (Kruskal Wallis test, Dunn-Šidák corrections, *p<0.05, **p<0.0001). After protein lysis and release, buffer exchange eliminates imidazole that interferes with UV-activated protein photocapture. Each black dot represents an endogenous protein from individual cells per microwell. Red lines indicate median with range from maximum to minimum expression values. β-TUB: n_{0M}=33, n_{1M}=66, n_{Buffer}_{_}_{Exchange}=141; CK8: n_{0M}=46, n_{1M}=116, n_{Buffer}_{_}_{Exchange}=328; GAPDH: n_{0M}=41, n_{1M}=69, n_{Buffer}_{_}_{Exchange}=201.

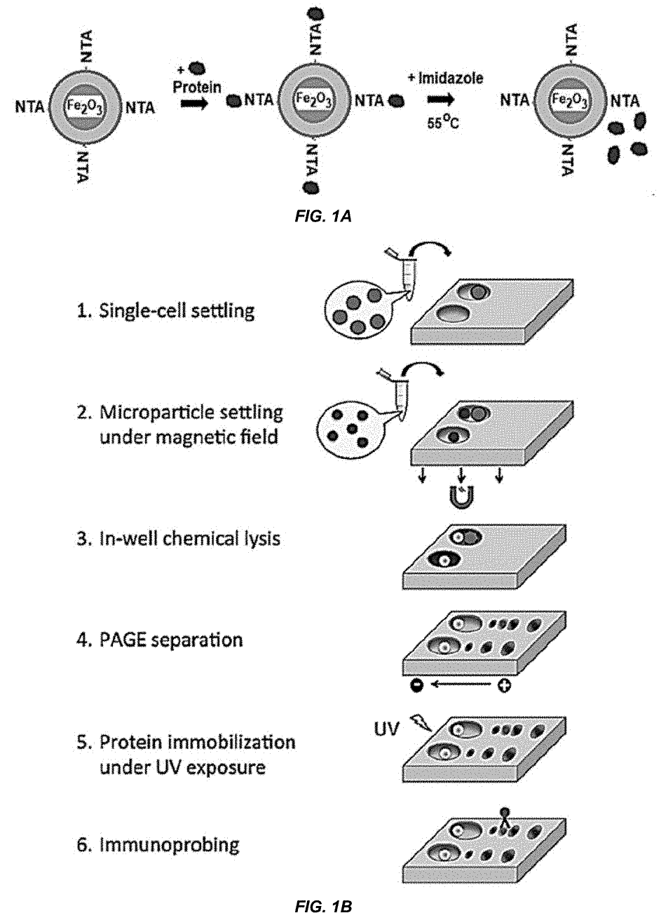

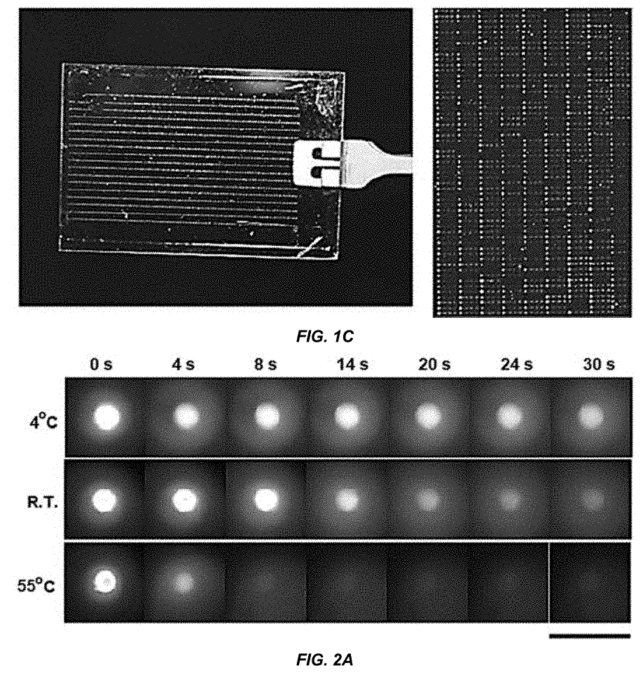

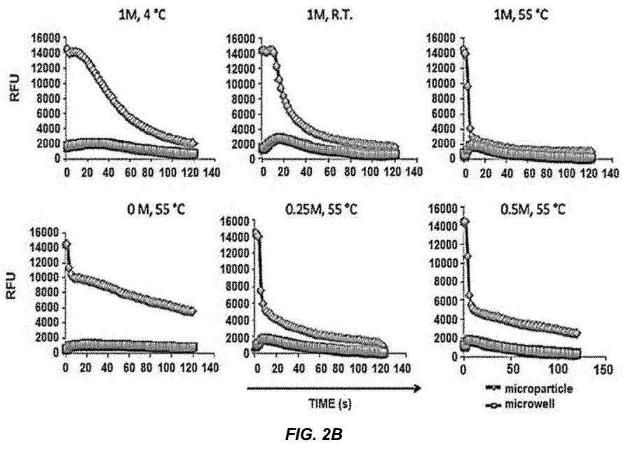

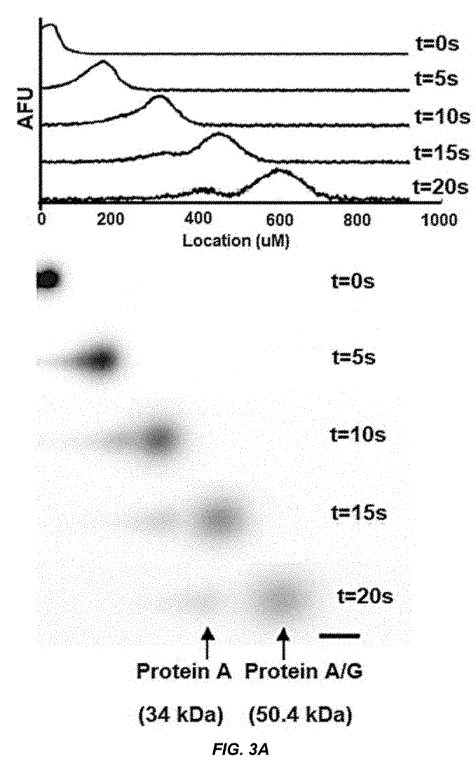

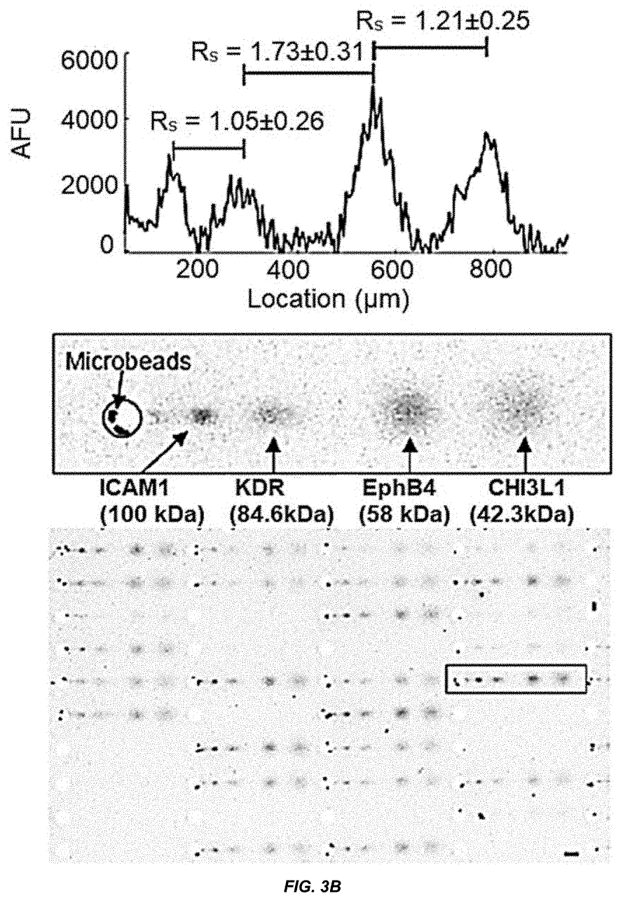

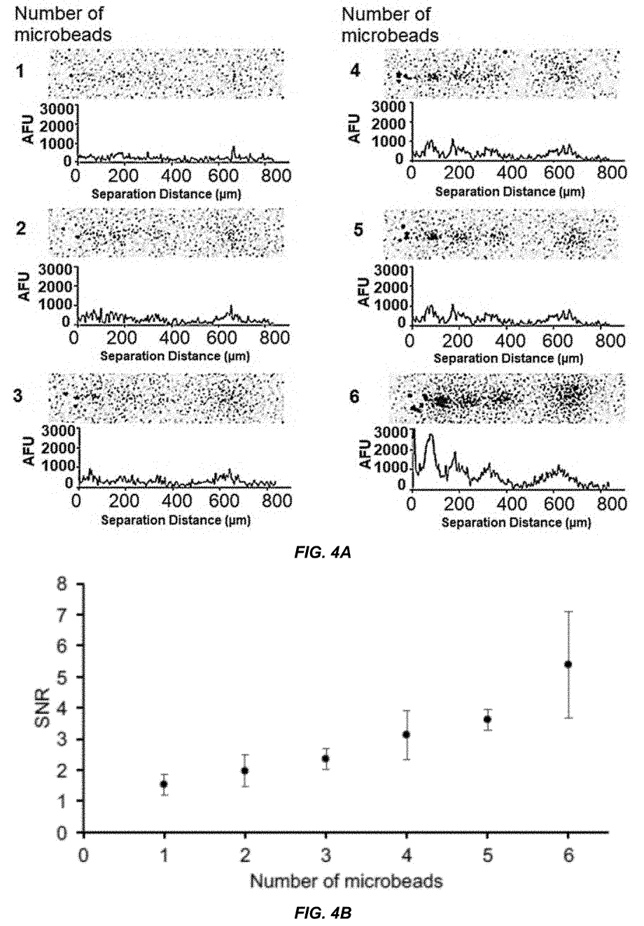

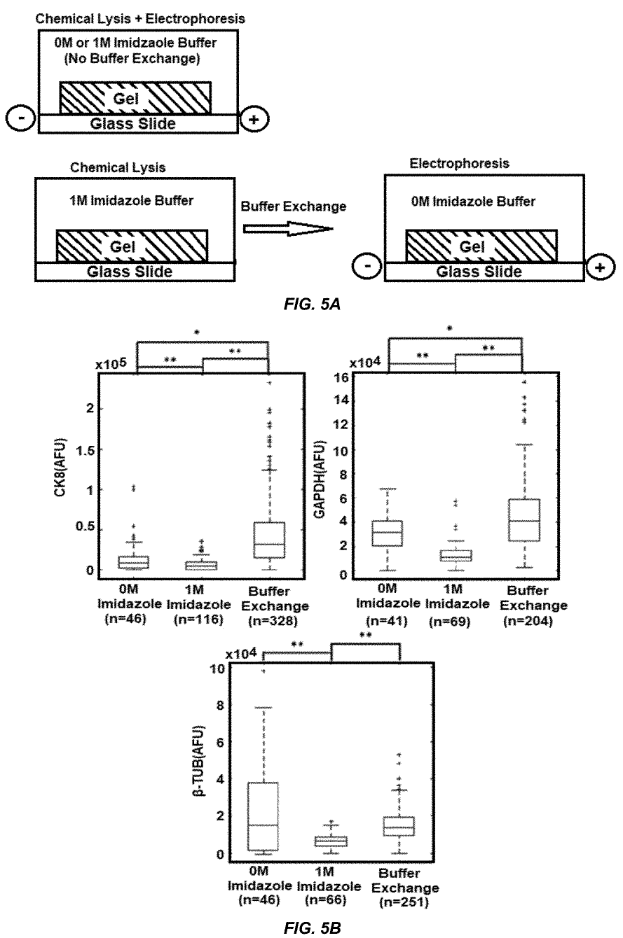

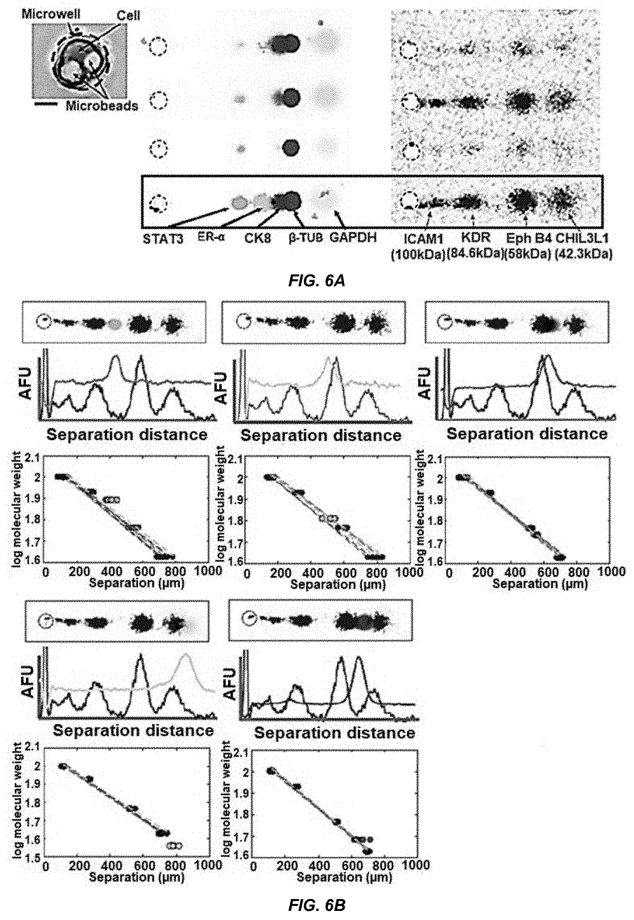

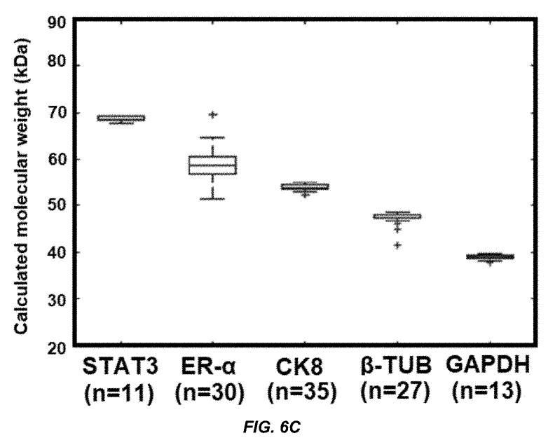

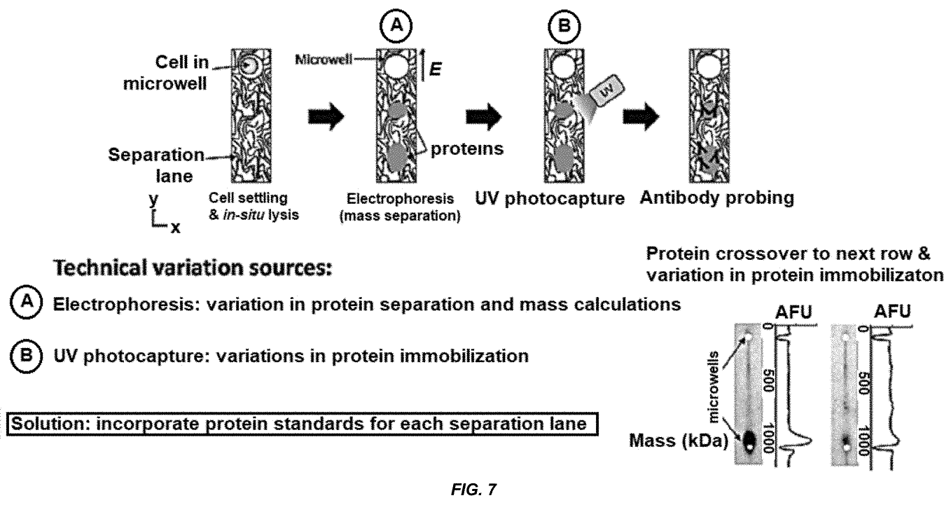

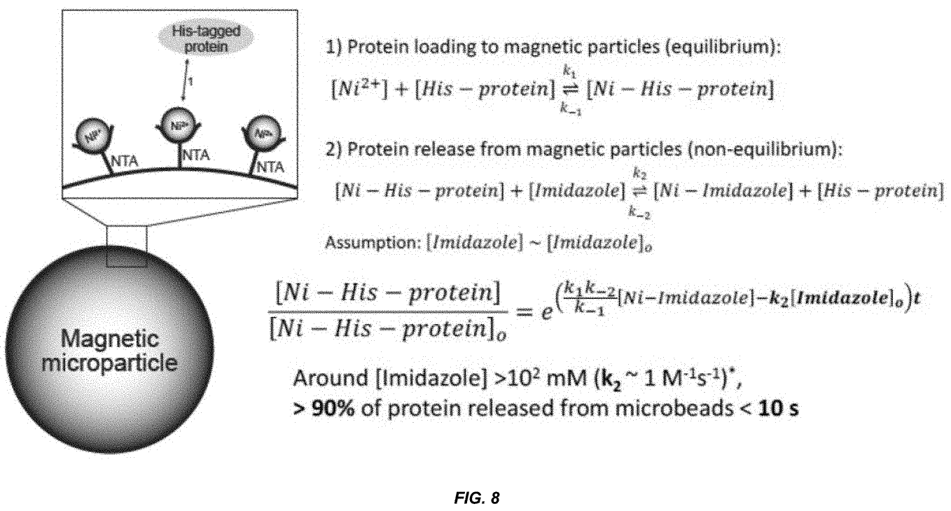

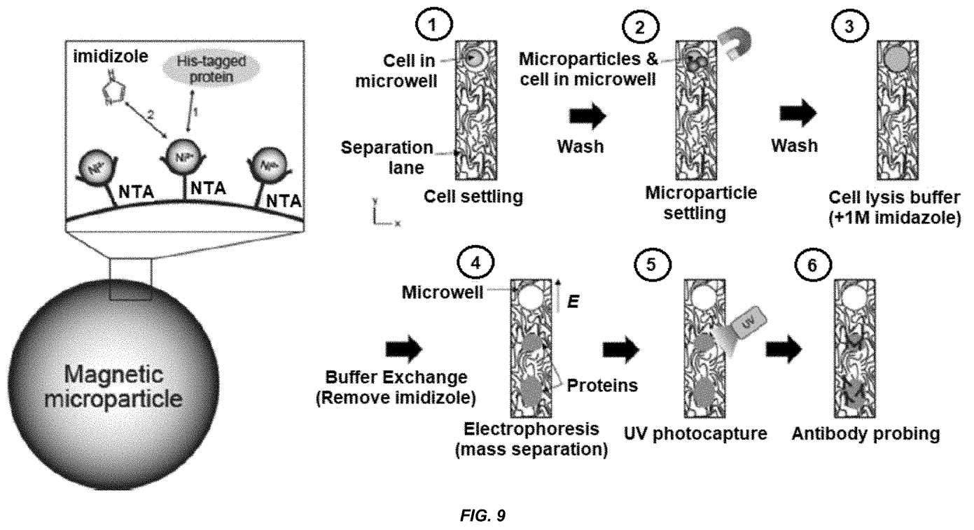

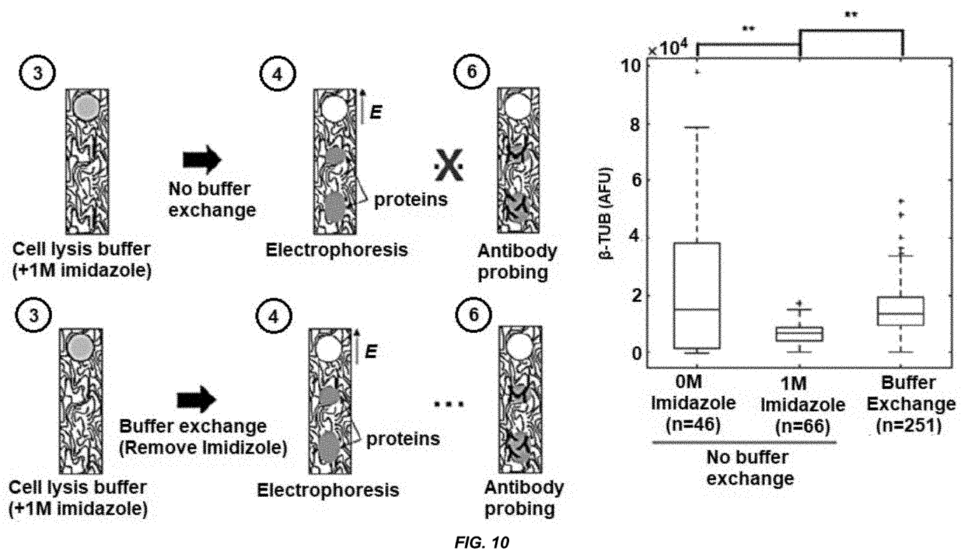

### DETAILED DESCRIPTION

As used herein and in the appended claims, the singular forms “a,” “an,” and “the” include plural referents unless the context clearly dictates otherwise. Thus, for example, reference to “an imidizole” includes a plurality of such imidazoles and reference to “the microparticle” includes reference to one or more microparticles and equivalents thereof known to those skilled in the art, and so forth.

Also, the use of “and” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. Moreover, with respect to any term that is presented in one or more publications that is similar to, or identical with, a term that has been expressly defined in this disclosure, the definition of the term as expressly provided in this disclosure will control in all respects.

Ranging from deep RNA sequencing to protein analysis, high-throughput microarray and cytometry has made it possible to analyze gene/protein heterogeneity at a single-cell resolution. Single-cell analysis offers powerful capabilities of identification of rare subpopulations of cells, understanding intra- and inter-heterogeneity of cancer, and tracking cell differentiation and reprogramming. Despite great potentials for uncovering new biological systems and targeting diseases with precision medicine, single-cell approaches are composed of complex device processes that can cause bias in measurement.

In deep sequencing, technical variation in single cell expression data occurs during capture and pre-amplification steps. To reduce technical variance and delineate from biological variance in single-cell RNA sequencing, external controls have been developed. Synthetic spike-ins and unique molecular identifiers (short-random DNA sequence) are used as RNA standards to directly measure sequencing error rates, sensitivity, and protocol and sample preparation biases. Similarly, in single-cell protein assays, technical variability can obscure functionally relevant variance. For example, protein capture is a process where technical variation can arise in assays. Protein capture variability is checked by spiking a buffer with large quantities of standard proteins or using a computational method (e.g., Monte Carlo simulation), but both methods are resource-intensive and dependent on specific platform. Alternatively, cells from a same batch or culture dish can be used to test the microarray with replicates. However, other non-functional biological variabilities, such as cell cycle and size, can impact functionally relevant protein measurement.

The disclosure provides methods and compositions for controlling protein measurement quality in single-cell assays. The disclosure provides micro- or nano-particles that can be used as vehicles for loading and releasing a protein standard(s). In a particular embodiment, a surface of a micro- or nano-particles is modified and functionalized to capture target proteins at specific concentrations. Chelation-assisted click chemistry is applied to demonstrate that protein standards with different molecular masses can be loaded and analyzed in a single-cell protein assay. Micro- or nano-particles are introduced into single-cell devices by either passive gravity, magnetic attraction, or other physicochemical forces. These protein standards from micro- or nano-particles provide a reference to measure protein mass sizes from individual cells and a quality control for any biases in device fabrication, cell lysis, protein solubility, protein capture, and protein readouts (i.e., antibody probing). Presented herein are the fabrication and experimental methods to make micro- or nano-particles that can be functionalized with click chemistry to reversibly append or reversibly attach tracer biomolecules to the micro- or nano-particles. Further provided herein are methods of using such micro- or nano-particles as protein standards in various microfluidic devices (e.g., a single-cell cytometry).

The micro- or nano-particles can be any particle to which a protein or tracer molecule can be reversibily bound. The particle can be a metal or non-metal. The particle can be spherical or any geometry. The particle can be between 10 nm to 999 um in cross section (e.g., diameter, length or width). In certain embodiments, the particle is magnetic.

In one embodiment, the disclosure provides for micro- or nano-particles that have been functionalized with nitrilotriacetic acid (NTA). For purpose of this disclosure, NTA-functionalized micro- or nano-particles refers to NTA that has been further complexed with nickel, cobalt, copper or other suitable metal ions, unless specified otherwise. Nitrilotriacetic acid (NTA) was first developed for immobilized metal affinity chromatography (IMAC) back in the 1980s. IMAC works by immobilizing chelating chemical groups on a substrate, which in turn chelates a bivalent metal ion (typically Ni⁺², Cu⁺², or Co⁺²). The bond strength for these types of interactions have been estimated to be in the 200-400 pN range by using single molecule AFM studies. Studies have been further performed to vary the number of histidine residues on the protein as well as the valency of NTA on the capture substrate in order to tune dissociation constants and thus provide a pathway for changing release kinetics. The metal complex binds sequential histidine residues on proteins and interacts with a high affinity ranging from 13 mM to 1.2 nM depending on NTA valency. Recently, NTA chemistry has been employed for a variety of new applications outside chromatography. Polystyrene micro- or nano-particles have been modified with NTA for flow cytometric analysis of proteins. NTA has also been used in lipid bilayers to examine protein interactions; 1,2-dioleoyl-sn-glycero-3-[N-(5-amino-1-carboxypentyl) iminodiacetic acid] succinyl (DOGS-NTA), a lipid conjugate of NTA, was used to create two-dimensional protein crystals and study protein-protein interactions on supported lipid bilayers. Researchers have functionalized liposomes with DOGS-NTA for capturing and presenting His₆-tagged proteins. In addition, poly(ethylene glycol) hydrogels have been functionalized with NTA in order to retard the release rates of proteins encapsulated in hydrogels.

In one embodiment, a tracer biomolecule is reversibly attached to the NTA functionalized micro- or nano-particles by using chelation click chemistry. A tracer biomolecule refers to a biomolecule that can be traced or tracked once released from the NTA functionalized micro- or nano-particles. Generally, a tracer biomolecule refers to a protein or peptide of known composition (e.g., weight, fluorescence, relative mobility etc.). In other instances, a tracer biomolecule can refer to other biomolecules like ribozymes, peptoids or hybrid molecules which comprise a protein or peptide that is bound to another biomolecule (e.g., DNA bound to a protein transcription factor). In a further embodiment, the tracer biomolecule can comprise a poly histidine tag (i.e., 4 or more histidine amino acids linked together). In particular embodiment, the tracer biomolecule comprises a 6× histidine tag. Poly-His tags have great affinity to NTA functionalized micro- or nano-particles and bind best at or near-neutral buffer conditions (physiologic pH and ionic strength). In one embodiment, the tracer molecule(s) comprise a plurality of peptides/polypeptides of different molecular weight having a predetermined relative mobility shift in polyacrylamide. In another embodiment, the tracer molecule(s) comprise a radioisotope or fluorescent/luminescent tag.

In another embodiment, the disclosure provides an NTA functionalized micro- or nano-particle (e.g., a metal-micro- or nano-particle comprising Nickel, Cobalt or Copper). The NTA functionalized micro- or nano-particle can be conjugated via the NTA moiety to His-tagged tracer molecule(s) (e.g. a His-tagged polypeptide(s)). In one embodiment, the His-tagged tracer molecules are of known molecular mass or ranges of molecular masses, having a predicated mobility shift that can be used as molecular weight markers or relative mobility shift markers. The His-tagged tracer polypeptide(s) can be incubated in a neutral buffer with the NTA functionalized micro- or nano-particles to obtain micro- or nano-particles conjugated via the NTA to the tracer polypeptide. Thus, in another embodiment, the disclosure provides micro- or nano-particles conjugated to tracer molecules via a linker.

During use, the tracer molecules can be dissociated from the NTA moiety by heating the micro- or nano-particle that is conjugated to the tracer molecule(s) (e.g., polypeptide(s)) to “release” the tracer molecule(s) from the micro- or nano-particle. In another embodiment, the tracer molecule(s) can be “released” from the micro- or nano-particle in the presence of high concentrations of imidazole, which competes for the NTA moiety thus displacing the tracer molecule(s).

In another embodiment, the disclosure provides a method of analyzing protein content in a sample, comprising capturing a sample in a microwell of a polyacrylamide fluidic device; optionally washing the fluidic device with a wash buffer; delivering a micro- or nano-particle conjugated to tracer molecule(s) to the microwell; optionally washing the fluidic device with a binding or wash buffer; lysing the sample and releasing the tracer molecules from the micro- or nano-particle with a lysis and release buffer; performing electrophoresis; and immunoprobing, blotting or staining the polyacrylamides gel fluidic device.

A typical binding/wash buffer consists of Tris-buffer saline (TBS) pH 7.2, containing 10-25 mM imidazole. The low-concentration of imidazole helps to prevent nonspecific binding of endogenous proteins that have histidine clusters. High concentrations of salt and certain denaturants (e.g., chaotropes such as 8 M urea) are compatible, so purification from samples in various starting buffers is possible, including buffer used to lyse cells. Release of the attached tracer biomolecule from the NTA-functionalized microparticle can be brought about by use of a completive ligand and/or with heating (e.g., imidiazole and/or heating at 55° C. or greater) For example, a high concentration of imidazole (at least 200 mM), low pH (e.g., 0.1 M glycine-HCl, pH 2.5) or an excess of strong chelators (e.g., EDTA) can be used.

The disclosure provides a molecular mass standard for single-cell western blotting. The disclosure provides micro- or nano-particles comprising protein markers delivered to microwells in ‘solid-phase’ as a coating on the micro- or nano-particle (e.g., a magnetic micro- or nano-particle) vehicle. In one embodiment the micro- or nano-particle can be magnetically directed and chemically triggered to release a protein marker for concurrent analysis of the protein marker with each electrophoretic separation of single-cell lysate. To assess intra- and inter-assay variability, the disclosure describes the use of His-tagged ligands, which allows customization of the protein markers to match the single-cell western target needs. Endogenous protein targets are identifiable with selectivity greater than immunoassays alone, owing to dual measurements of molecular mass and reactivity with an immunoprobe.

The methods and compositions of the disclosure provide protein markers to aid in identification of unknown protein targets from single cells, as well as very importantly aiding in the identification and subsequent measurement of sources of technical variation in single-cell western blotting. Inclusion of protein markers allows a user to understand the variation arising from each stage of their custom assay—from cell lysis to immunoprobing—which allows adjustment of assay conditions to modulate the dominant contributors to variation (e.g., peak location, area-under-the-curve, dispersion).

Design and development of protein markers that are compatible with other single-cell immunoblotting modalities—including immunoprobed isoelectric focusing and immunoblotting of native species—is also provided. With careful design to deliver controlled (and known) quantities of standard proteins to each microwell, protein markers have the potential to aid in absolute protein quantitation of targets from each single-cell lysate and possibly even from sub-cellular compartments.

The following examples are intended to illustrate but not limit the disclosure. While they are typical of those that might be used, other procedures known to those skilled in the art may alternatively be used.

### EXAMPLES

### Example 1

Preparation of Fe₃O₄ Magnetic Microparticles.

FeCl₃ (1 M) and FeCl₂ (0.5 M) are dissolved in aqueous hydrochloric acid (0.4 M, 10 mL) at room temperature under sonication. After the salts are completely dissolved in solution, the mixture is degassed using a pump. Aqueous sodium hydroxide (0.5 M, 100 mL) is slowly added under nitrogen with stirring at room temperature. The mixture is left to react, heated in an oil bath at 80° C. for 30 min. After cooling to room temperature, the magnetic microparticles are rinsed with aqueous hydrochloric acid (0.1 M) and ethanol to remove any unreacted impurities followed by resuspension in deionized water (40 mL) under sonication for 30 min. The supernatant is collected after centrifugation at 13,500 rpm for 30 min at 4° C.

Preparation of NTO Functionalized Fe₃O₄ Magnetic Microparticles.

The supernatant, containing iron oxide magnetic microparticles (0.3 mg/mL, 100 mL), is mixed with ethanol (400 mL) and put in a water bath at 40° C. TEOS (0.375 mL) is added and mixed well, followed by slowly adding aqueous ammonia solution (10%, 20 mL) with stirring. After stirring for 12 h, the mixture is centrifuged at 13,500 rpm for 30 min and the supernatant was discarded. The microparticles remaining in the vial are rinsed with ethanol and methanol (280 mL) and resuspended in methanol (200 mL) under sonication for 30 min. The suspension is degassed using a pump followed by covering with nitrogen gas. The suspension is then heated in an oil bath at 110° C. with reflux before the addition of EDAS solution (80%, 2 mL) with stirring. After 12 h, the mixture is centrifuged at 13,500 rpm for 25 min. The supernatant is removed, and the isolated particles are rinsed with methanol. The particles are resuspended in DMF (50 mL) under sonication for 30 min. The suspension is stirred with succinic anhydride (0.2 g/mL, 10 mL) dissolved in DMF under nitrogen for 10 h. The mixture is centrifuged at 13 500 rpm for 25 min, and the supernatant is removed. The microparticles remaining in the vial are resuspended in methanol under sonication for 30 min followed by centrifugation at 13,500 rpm for 25 min. The supernatant is removed, and the rinsing is repeated three times. The rinsed microparticles are resuspended in deionized water under sonication for 30 min followed by centrifugation at 13 500 rpm for 25 min. This rinsing is repeated twice. The microparticles are resuspended in deionized water (40 mL) under sonication for 30 min. The suspension (20 mL) is mixed with EDC (100 mg) and NHS (100 mg) with stirring for 30 min, followed by the addition of NTA derivative (10 mg). The mixture is left to react at room temperature for 24 h and centrifuged at 13 500 rpm for 30 min. The supernatant is removed, while the isolated particles (Fe₃O₄/NTA) are rinsed with deionized water three times and resuspended in deionized water (20 mL).

Immobilizing Ni(II) Ions onto the Surface of Fe₃O₄/NTA Magnetic Microparticles.

The microparticles (120 μg) are vortex-mixed in aqueous nickel chloride solution (0.1 M, 200 μL) for 1 h. The solution is removed by magnetic separation, and the microparticle-Ni(II) conjugates (Fe₃O₄/NTA-Ni(II)) are rinsed with deionized water (200 μL×3) and resuspended in deionized water (200 μL) before use. The concentration of the micro- or nano-particle in the suspension was ˜0.6 μg/μL. When estimating the binding capacity of Ni(II) ions on the microparticles, the Fe₃O₄/NTA-Ni(II) microparticles (120 μg) in the suspension is collected by magnetic separation and the microparticles are resuspended in an EDTA solution (0.125 mg/mL, 100 μL) in methanol/0.05% ammonia (1/1, v/v) solution, with vortex mixing for 30 min. The solution, containing the EDTA-metal ion complex from the particle suspension, is collected for analysis by electrodeless/sheathless ESI MS.29,30 The nickel(II) is determined using a calibration curve obtained from ESI MS analysis

Preparation of Silica and Polystyrene Microspheres Functionalized with Ni-NTA.

NTA ligand (N-(5-amino-1-carboxypentyl)iminodiacetic acid) bearing a primary amine group is covalently attached to carboxylated polystyrene or silica microspheres. Carboxylated microspheres are washed, resuspended to 2×10⁸ microspheres/ml in 2-(N-morpholino) ethanesulfonic acid buffer (0.05 M; pH 6.5), and then activated (10 min, 22° C., agitation) by adding 0.1 mg NHS and 1 mg of EDC per 1×10⁸ microspheres. Activated microspheres are centrifuged and resuspended to 1×10⁸ microspheres/ml in HEPES (0.1 M, pH 8.0). 1 mg of EDC and 0.1 mg of NHS is added to 1×10⁸ microspheres before the primary amine group containing NTA ligand was added (1-5×10¹⁰ molecules NTA ligand per microsphere). After incubation for 18 h at 22° C., microspheres are centrifuged, residual free carboxy groups were inactivated by resuspending the microspheres in Tris-buffer (0.05 M; pH 8), and microspheres are washed two times in phosphate buffered saline (PBS; pH 7.4) by repeated cycles of centrifugation (14,0000 rpm, 5 min) and resuspension. Microspheres are incubated with NiCl₂ solution (500 μM, 2 h, 22° C.). After loading, microspheres are washed three times in PBS, resuspended to 5×10⁷ microspheres/m in PBS and stored at 4° C. Microspheres are stable for at least 1 year when stored under these conditions. After coupling of the NTA ligand, all buffers used for polystyrene microsphere preparations contained 0.005% of Tween 20.

Attachment of 6× His Tagged Proteins to NTA Functionalized Magnetic Microparticles.

Poly-His tags bind best to IMAC resins in near-neutral buffer conditions (physiologic pH and ionic strength). A typical binding/wash buffer consists of Tris-buffer saline (TBS) pH 7.2, containing 10-25 mM imidazole. A series of proteins were attached to NTA-functionalized microparticles disclosed herein (see Table 1).

TABLE 1
List of proteins carried by NTA coated magnetic microparticles.

| | | Mw | Company (Catalog |
|---|---|---|---|
| Protein | Species | (kDa) | no.) |
| Recombinant Protein A, His Tag | E. coli | 39 | Abcam (ab52953) |
| CHI3L1 recombinant mouse | Mouse | 42.3 | Thermofisher |
| protein, His tag | | | (50929M08H50) |
| Recombinant Protein A + Protein | E. coli | 59.7 | Abcam (ab52213) |
| G, His Tag | | | |
| Recombinant human Eph receptor | Human | 58 | Abcam (ab167746) |
| B4 protein | | | |
| KDR (VEGFR2) Recombinant | Human | 84.6 | ThermoFisher |
| Human Protein, His Tag | | | (10012H08H50) |
| ICAM1 Recombinant Human | Human | 100 | ThermoFisher |
| Protein, hIgG1-Fc. His Tag | | | (10346H03H25) |

### Example 2

Chemicals/Reagents:

Acrylamide/bis-acrylamide (40% wt/wt) solution (A7802), N,N,N′,N′-tetramethylethylenediamine (TEMED, T9281), ammonium persulfate (APS, A3678), sodium deoxycholate (D6750), β-Mercaptoethanol (M3148), imidazole (792527), and sodium dodecyl sulfate (SDS, L3771), were obtained from Sigma-Aldrich. Triton X-100 was purchased from Fisher Scientific (BP-151). 10× Tris/glycine buffer was obtained from Bio-Rad (161-0734). PBS, pH 7.4 was obtained from Gibco (10010-023). Tris-HCl, pH 6.8 was purchased from Teknova (T1568). PureProteome nickel magnetic microparticles with 10-μm diameter was obtained from Millipore Sigma (LSKMAGH02). A 6-tube magnetic separation rack was obtained from New England BioLabs (S1506S). N-[3-[(3-Benzoylphenyl)-formamido]propyl] methacrylamide (BPMAC) was custom synthesized by PharmAgra Laboratories. SU-8 developer (Y020100) and photoresist SU-8 2025 (Y111069) were obtained from MicroChem. Deionized water (ddH2O, 18.2 mΩ) was obtained using ultrapure water system (Millipore). Unless stated otherwise, chemicals and reagents were obtained from Sigma Aldrich.

Proteins:

Recombinant protein A His Tag (Protein A, ab52953) and recombinant human EpH receptor B4 protein His Tag (EpH, ab167746) were obtained from Abcam. Protein A and EpH are fused with 6× polyhistidine domains on N- and C-terminus, respectively. VEGFR2 (KDR) recombinant human protein, His Tag (KDR, 10012H08H50), ICAM1 recombinant human protein, hIgG1-Fc. His Tag (ICAM1, 10346H03H5), and CHI3L1 recombinant mouse protein His Tag (CHI3L1, 50929M08H50) were purchased from ThermoFisher Scientific. KDR, ICAM1, and CHI3L1 are expressed from a DNA sequence from the extracellular domains fused to a C-terminal polyhistidine tags. Unless stated otherwise, His Tag proteins were fluorescently labelled using Alexa Fluor 647 NHS ester succinimidyl ester (Life Technologies, A20006). Fluorescently labelled His Tag proteins were purified using dye removal columns (ThermoFisher Scientific, 22858) according to the manufacturer protocol. Details of the recombinant proteins used for protein markers are listed in Table 2.

TABLE 2
List of protein carried by Ni-conjugated
magnetic micro-particles.

| | | Mw | Company |
|---|---|---|---|
| Protein | Species | (kDa) | (Catalog no.) |
| Recombinant Protein A, His Tag | E. coli | 39 | Abcam (ab52953) |
| CHI3L1 recombinant mouse | Mouse | 42.3 | Thermofisher |
| protein, His tag | | | (50929M08H50) |
| Recombinant Protein A + Protein | E. coli | 59.7 | Abcam (ab52213) |
| G, His Tag | | | |
| PDGFRA recombinant human | Human | 57.7 | Thermofisher |
| protein (without catalytic activity), | | | (10556H08H25) |
| His tag | | | |
| Recombinant human Eph receptor | Human | 58 | Abcam (ab167746) |
| B4 protein | | | |
| KDR (VEGFR2) Recombinant | Human | 84.6 | ThermoFisher |
| Human Protein, His Tag | | | (10012H08H50) |
| ICAM1 Recombinant Human | Human | 100 | ThermoFisher |
| Protein, hIgG1-Fc. His Tag | | | (10346H03H25) |

Primary antibodies to recognize endogenous proteins include rabbit anti-β-tubulin (ab6046, Abcam), goat anti-GAPDH (SAB2500450, Sigma), mouse anti-cytokeratin 8 (C5301, Sigma), rabbit anti-estrogen receptor α (ab16660, Abcam), rabbit anti-STAT3 (79D7, Cell Signaling). For the primary antibody host species, secondary antibodies with Alexa Fluor were purchased from ThermoFisher Scientific: anti-mouse secondary antibody with Alexa Fluor 555 (A31570), anti-rabbit secondary antibody with Alexa Fluor 488 (A21206), anti-goat secondary antibody with Alexa Fluor 555 (A21432).

Protein Loading on Nickel Microparticles:

Magnetic microparticles (5 μl) were equilibrated with buffer (50 mM sodium phosphate, 300 mM sodium chloride, 10 mM imidazole, pH 8). A protein solution (500 μl) containing a mixture of His Tag proteins in (30% v/v) ethanol/PBS was loaded with the microparticles and mixed gently for 2 h at 4° C. using a rotator. Un-bound proteins were washed 3 times using wash buffer (50 mM sodium phosphate, 300 mM sodium chloride, 20 mM imidazole, pH 8). Residual liquid was separated from the microparticle using a magnetic rack and removed after each of the above steps. The protein bound microparticles were resuspended in 1×PBS.

In order to determine protein release kinetics, microparticles (5 μl) was loaded with Protein A (4.1 μg). For electrophoretic separations, microparticles (5 μl) was loaded with either one of two protein mixtures containing: (1) Protein A (2 μg), or (2) CHI3L1 (14.8 μg), EpH (54.5 μg), KDR (21.9 μg), and HIgG1-F (7.4 μg). The bound His Tag protein was eluted from the microparticles using lysis/electrophoresis buffer prepared with imidazole (0 or 1 M), SDS (2.5 g), sodium deoxycholate (1.25 g), Triton X-100 (500 μl), Tris/glycine buffer (25 ml, 10×) and ddH₂O (474.5 ml).

Measurement of Microparticle Loading Efficiency Via Flow Cytometry:

The percentage of microparticles loaded with AF647-labeled proteins was determined by flow cytometry. The fluorescence (RL1-A, with PMT gain 180) of microparticles with and without the protein ladder was measured using an Attune N×T acoustic focusing flow cytometer (ThermoFisher Scientific). Microparticles were drawn from a solution (333,333 microparticles/ml), and a threshold of FSC-A>25000 was applied to avoid detection of debris. Results were analyzed using FlowJo analysis software (FlowJo).

Cell Culture:

The MCF-7 breast cancer cell line was purchased from ATCC and maintained in RPMI 1640 (ThermoFisher Scientific) containing fetal bovine serum (10%) and penicillin/streptomycin (1%) in a humidified incubator held at 37° C. under 5% CO₂. MCF-7 was tested mycoplasma negative and authenticated with short tandem repeat analysis.

Fabrication of Single-Cell Western Blot Chip:

The master mold comprised a silicon wafer with SU-8 features that was fabricated according to standard photolithography procedure. Single-cell western blot devices containing an array of microwells (250 μm well-to-well spacing and 1 mm long separation lane) with feature heights and diameters of 35 μm and 30 μm, respectively, were fabricated by casting a polyacrylamide gel against the mold.^{[46]} The polyacrylamide gel layer was chemically polymerized (7% T, 3.45% C, 3 mM BPMAC, 0.08% APS and 0.08% TEMED).

Single-Cell Western Blot Buffer Exchange:

After settling cells in microwells by gravity, a chemical lysis buffer (0.5× Tris glycine, 0.5% SDS, 0.25% sodium deoxycholate, 0.1% Triton X-100, 1 M Imidazole, pH 9.2, 55° C.) was poured into the single-cell western blot for 30 s to release protein markers from microparticles and solubilize endogenous proteins from mammalian cells. Then, a 2 s electrophoresis at 40 V/cm was applied immediately for protein injection. For protein separation, an electrophoretic buffer (0.5× Tris glycine, 0.5% SDS, 0.25% sodium deoxycholate, 0.1% Triton X-100, pH 8.7, 22° C.) was introduced, followed by immediate electrophoresis at 40 V/cm for 23 s.

Fluorescence Imaging:

Protein A bound microparticles were prepared and settled into microwells under a magnetic field. Fluorescence protein release from the microparticles in the microwells were visualized using an Olympus IX71 inverted fluorescence microscope equipped with ASI motorized stage, X-cite mercury lamp light source (Lumen Dynamics) and standard Cy5 filter cube (40× objective). Time-lapse images were captured using an iXon+ EMCCD camera (Andor Technology Ltd) controlled by a MetaMorph software (Molecular Devices) with 60 ms exposure time and 2 s time intervals.

Image Analysis and Quality Control:

For the release kinetics measurement, image processing was performed using a Python script (Anaconda Python 3.5.3). Circular region of interest (ROI) were manually selected via a Graphical User Interface provided using the cv2 Python package (OpenCV 3.1.0). Image pixel statistics were estimated using the numpy Python package (numpy 1.13.1). Mean fluorescence intensity from a ROI covering the whole microparticle with AF647-labeled Protein A was obtained using the widefield fluorescence microscope, which accounted the out-of-focus fluorescence along the z-axis. Background signal was designated as the mean fluorescence intensity measured from an adjacent empty microwell. Each mean fluorescence intensity of the microwell was background subtracted and then normalized to the value at the start of the release process (t=0).

For the single-cell western blot analysis, images of proteins were collected by scanning the single-cell western blot devices with a fluorescence microarray scanner (Genepix 4300A, Molecular Devices). The images were processed by applying a median filter with a 2-pixel radius and a threshold value of 50 (ImageJ). Quantitation of proteins from the images was processed by in-house MATLAB (R2016b) scripts. Protein peaks were fitted with Gaussian functions. For quality control protocol, the protein peaks with Gaussian fitting R²≥0.7 and SNR>3 were analysed. From the curve fitting, protein peak width and location were extracted to determine protein molecular mass from Ferguson analysis.

Design of the Rapid-Release Protein Marker Delivery Vehicle.

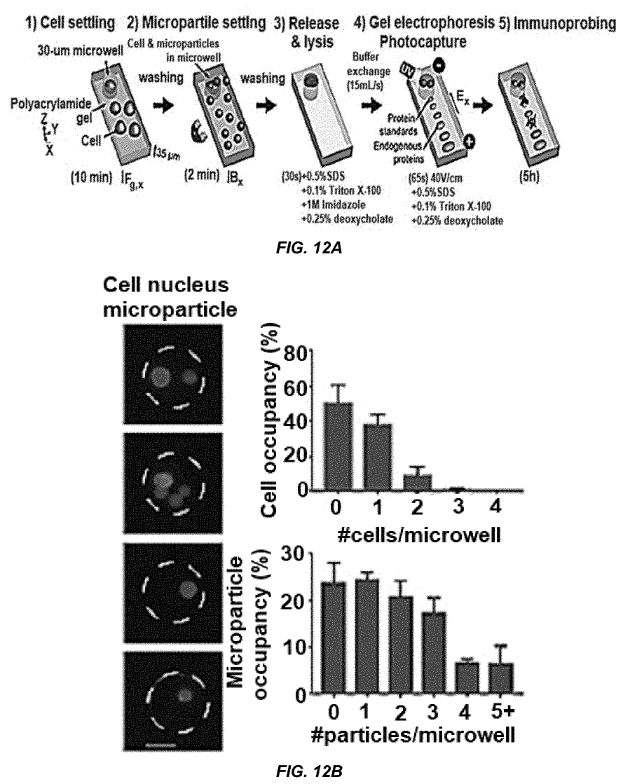

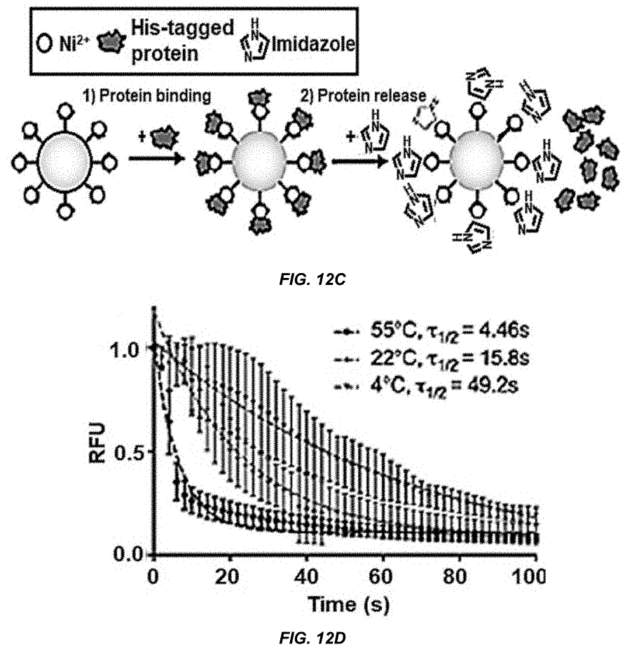

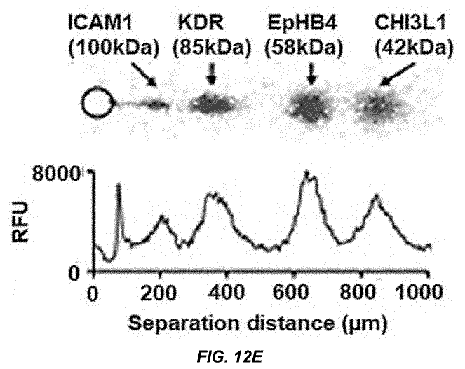

A magnetic microparticle delivery system was selected for delivery of protein markers to each microwell of the multi-stage, single-cell western blot (FIG. 12A). The method used: (i) an applied magnetic field to actively seat marker-coated microparticles in microwells (i.e., in contrast to gravity-based cell settling; FIG. 12B) and (ii) a flexible coordination chemistry on the microparticle surface for protein marker immobilization and triggered solubilization/release (FIG. 12C). Two key microparticle characteristics were used to achieve the desired functionality and performance: (i) magnetic cores for active microparticle seeding in microwells directed via magnetic field and (ii) a Ni²⁺ coating that supports high-efficiency protein immobilization with rapid protein release (<30 s) when exposed to imidazole.

The experiments first sought to assess the efficiency of a two-step microwell loading process. First, MCF-7 breast cancer cells were seated in each microwell by applying a suspension of the cells (˜25 μm diameter) onto the polyacrylamide gel layer stippled with 1300 microwells. Cells sedimented via gravity for 10 min (FIG. 12A, step 1). Gentle washing removed cells that settled onto the surface of the polyacrylamide gel, with cells that settled into the microwells retained on the chip. Next, to deliver microparticles coated with the 4 protein markers into each cell-laden microwell, a solution of the microparticles (10⁶ microparticles/ml) were applied to the single-cell western blot device and applied a magnetic field to the device for 2 min to direct microparticles into microwells (FIG. 12A, step 2). The circular magnet (4 cm diameter×1.53 cm height) was located under the 2.5 cm×3.75 cm device and was moved across the device to expose all microwells to the applied magnetic field. Gentle washing was used to remove microparticles from the surface of the polyacrylamide gel, with microparticles that settled into the microwells retained on the chip.

At the completion of the two-stage microwell loading process, brightfield microscopy was used and a random region of interest was selected (˜300 microwells) from each chip to analyze both microparticle and cell occupancies. Approximately 75% of the microwells sampled housed≥1 microparticle (n=3 chips, standard deviation σ=4%, FIG. 12B). Microwells were designed to house MCF-7 cells (30 μm diameter and 35 μm deep microwells), a 10-μm diameter microparticles was selected so as not to interfere with cell loading. About 38.3% of microwells contained a single MCF-7 cell (FIG. 12B). In addition to passive cell sedimentation, alternate approaches for cell loading including, for example, precision transfer of rare cells and two-step centrifugation by utilizing a second layer of polyacrylamide gel as raised and crescent-shaped dam structures on the perimeter of each microwell can be used. A centrifugal force directed cells to the dams. Upon removal of the centrifugal force, each cell localized to the dam then settles into the proximal microwell. For the two-step loading process, 35.3% of microwells contained both a single cell and ≥1 microparticle (n=3 chips, ˜300 microwells sampled per chip, standard deviation σ=8.1%), as desired for concurrent electrophoretic analysis of protein markers and cell lysate from each microwell.

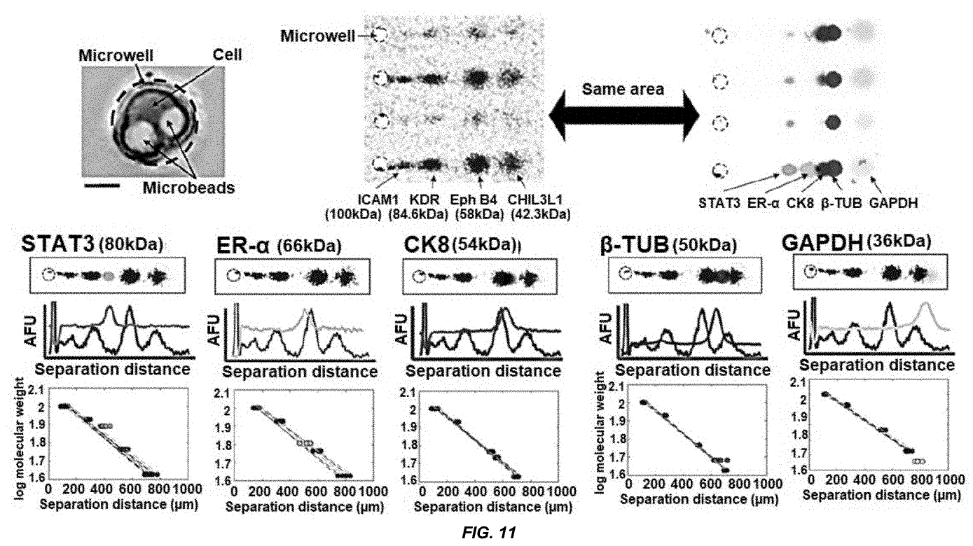

Experiments were then performed to quantitatively assess (i) the Ni²⁺ chelation for solid phase immobilization of protein markers on the microparticle surface and (ii) the subsequent rapid triggered solubilization of protein markers into the PAGE separation stage (FIG. 11C-D). Protein markers were released from the microparticle surface with a <30 s release duration and a ‘pulse’ release profile. The <30-s duration was sought to match the time frame of the rapid, in-microwell cell lysis and endogenous protein solubilization step. A pulse profile was sought to minimize injection dispersion (i.e., sample tailing) arising from marker release during electroinjection of microwell contents into the PAGE lane.

To immobilize the protein markers on the microparticle surface, the microparticles were incubated (2 h) in a cocktail of fluorescently labelled His-tagged marker proteins (KDR, 21.9 ng/μl; ICAM1, 7.40 ng/μl; EpH B4, 54.5 ng/μl; CHI3L1, 14.8 ng/μl). After incubation with proteins, the microparticles were washed 3 times with a washing buffer containing mild imidazole (20 mM). Via flow cytometry, about 98% of microparticles (˜2000 particles) were coated with the fluorescent protein markers (FIG. 12C).

After preparing the microparticle-based protein marker delivery system, the release kinetics of His-tagged recombinant Protein A labelled with Alexa Fluor 647 (AF647) was examined as a representative protein marker. To displace the His-tagged proteins from the microparticle surface, imidazole was used as a competitive binding ligand. Imidazole has a similar structure to His and serves as an electron donor. Release of His-tagged Protein A from the microparticles was modelled according to one-state ligand-receptor kinetics (an exponential decay).

Next, experiments were performed to assess the impact of imidazole concentration on protein marker release from the microparticles using a series of different imidazole concentrations in the cell lysis buffer. During the protein marker release from the Ni-His linkage it was assumed that the competitive binding of imidazole to the Ni²⁺ is not affected by the molecular size of protein markers conjugated to the His tag, given that the molecular mass of imidazole is 68 Da. The lowest imidazole concentration studied was 15 mmol imidazole, which was in excess as compared to the concentration of the His-tagged Protein A (0.12 nmol). Given the orders-of-magnitude excess of imidazole, it was assumed that the imidazole concentration in the buffer is not significantly reduced over the 30-s time course of protein marker release and cell lysis, even for this lowest imidazole concentration condition. Protein A was used as a model marker protein for the release because Protein A is a low-molecular mass marker (39 kDa) that enables measurement of the protein fraction bound on microparticles using wide-field fluorescence microscopy—the unbound protein diffuses away quickly from the region of interest (estimated diffusion coefficients, 36.1 μm²/s at 4° C. to 158 μm²/s at 55° C.). The Protein A release kinetics at the 1 M imidazole concentration and at 4° C. yield a protein marker release half-life of 49.2 s (95% confidence interval, CI=39.0-65.9 s) and a dissociation rate constant of 0.014 s⁻¹ (n=3 particles, coefficient of variation, CV=123.7%; FIG. 12D). For the same imidazole concentration, the temperature of the cell lysis buffer was elevated and the dissociation rate constants obtained was 0.044 s⁻¹ (at 23° C.; n=3 particles, CV=51.5%) and 0.16 s⁻¹ (at 55° C.; n=3 particles, CV=40.6%), with the latter condition yielding a protein release half-life of 4.46 s (95% CI=4.01-4.97 s). Within 30 s, more than 80% of the His-tagged Protein A was released from the microparticles at 55° C. (FIG. 12D). The dissociation rate constant was examined for a range of higher imidazole concentrations at the most elevated temperature condition. A short release half-life (4.46 s) was observed with a 1 M imidazole concentration at 55° C. Because the estimated time scale of the His-tagged Protein A (39 kDa) diffusing out of a microwell is similar to the protein release half-life in 55° C. 1 M imidazole, the release reaction is rapid compared to diffusion of protein out of the well. Any protein markers with ≥39 kDa would remain at higher concentrations in the microwell prior to single-cell protein PAGE. As the protein marker delivery would be concurrent with the rapid cell lysis step, 1 M imidazole at 55° C. was selected as the condition to trigger release of His-tagged protein markers.

Validating the Protein Marker as a Molecular Mass Standard.

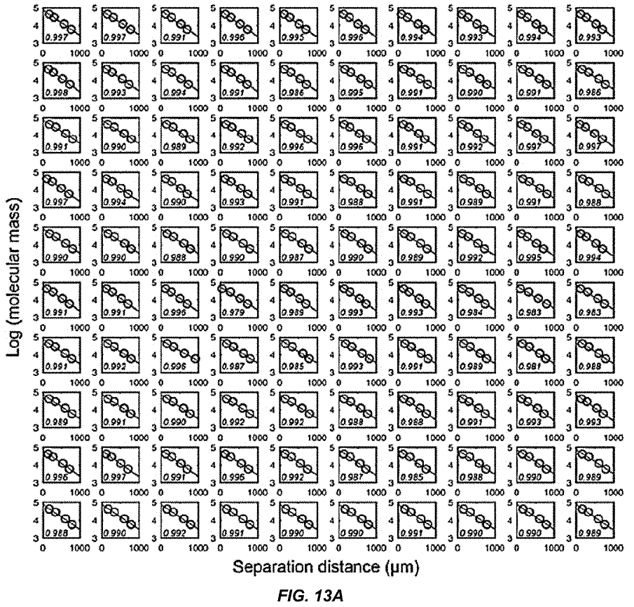

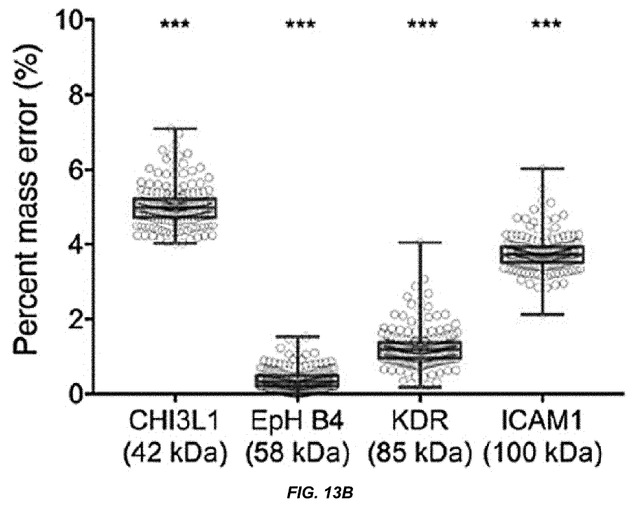

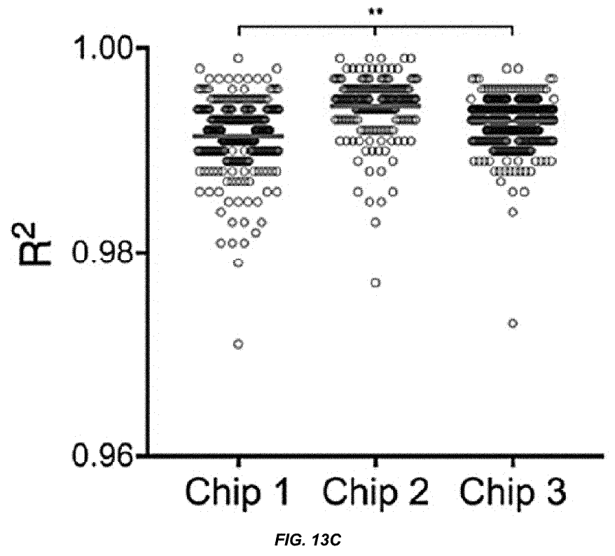

After rapid release of the protein markers in the microwell, The protein marker electromigration was analyzed during single-cell protein PAGE (FIG. 12E, 13). At 30-s of cell lysis and protein marker release, protein PAGE was initiated by applying an electric field (E=40 V/cm) for 25 s. Separated protein peaks were immobilized with a 45 s UV exposure of the benzophenone in the polyacrylamide gel. After photoblotting, Ferguson analysis of the protein electromigration was performed. In protein sizing (gel electrophoresis with SDS), a log-linear relationship between the molecular mass and observed electrophoretic mobility of each target was expected. Log-linear fitting of the protein markers yielded R²>0.97 for >300 PAGE separation lanes (FIG. 13A), which is comparable to the log-linearity of many other slab gel and capillary electrophoresis systems. The final protein peak location had CV ranging from 2.2% to 11.0% depending on the protein marker considered (μ±σ of CV across 3 chips: ICAM1, 9.41±2.06; KDR, 6.29±2.03; EpH B4, 5.00±2.30; CHI3L1, 5.07±2.53; n_{chip1}=147, n_{chip2}=104, n_{chip3}=340; Table 2).

Across each device, no observable spatial dependence on the y-intercept values of the log-linear regression was noted, but a decrease in the slope across the width of the chip was observed. Recognizing that a ΔE=˜1 V/cm across the width of the chip (left to right) would result in the observed slope variation; it was hypothesized that the apparatus has a slight offset in electrode pair spacing from one end of the chip to the other.

For each protein marker, the expected molecular mass was compared to the measured molecular mass. An analysis of 168 single-cell PAGE lanes gave a difference between the two values of <5%, with an interquartile range (IQR)<1% (FIG. 13B), for all protein markers that passed quality control. Although peak location CV values are not significantly different among all four markers (Table 2, n=3 chips, one-way ANOVA test, p>0.5), the percent mass error of the smallest ladder component (CHI3L1, 42 kDa) and the largest ladder component (ICAM1, 100 kDa) were significantly higher than those of EpH B4 (58 kDa) and KDR (85 kDa) (p±a of percent mass error: ICAM1, 3.77±0.49; KDR, 1.24±0.53; EpH B4, 0.39±0.30; CHI3L1, 5.05±0.57; Kruskal-Wallis test with Dunn's multiple comparisons test, ***p<0.0001, n=168 PAGE separation lanes; FIG. 13B). The slight higher mass errors in CHI3L1 and ICAM1 were ascribable to CHI3L1 and ICAM1 peaks electromigrating less than what the log-linear regression fitting expected, resulting in the estimated masses lower than the expected masses. High and low molecular mass proteins should be included in each set of protein marker standards for most accurate estimation of molecular mass, which agrees with previous work demonstrating that the use of a pair of high and low molecular mass protein standards controlled for technical variation in migration rate in a capillary gel electrophoresis platform.

Next, chip-to-chip variation was examined in molecular mass estimation to assess reproducibility of the protein marker as a size standard (FIG. 13C). Regarding the goodness of the log-linear regression fit, R² was observed to be >0.97 (n=3 chips; FIG. 13C). One-way ANOVA tests indicate that the distributions of the R² values are significantly different among the chips. However, the magnitude of the chip-to-chip difference in R² values is minimal, with a Cohen's d<0.2 (FIG. 13C). The technical variation across the chips was further examined by analyzing percent mass errors of the protein markers. Although the slight run-to-run difference in the gel electrophoresis duration might contribute to the fact that distributions of percent mass errors are significantly different across chips, consistent protein electromigration (CV of peak locations<12%) and <10% mass errors in sizing of the protein markers (42-100 kDa protein masses (Table 2) was observed.

Given that not all cell-containing microwells were populated with microparticles, the impact of protein marker performance was examined when employing protein markers proximal to single-cell PAGE separations lanes as size standards. A semi-variogram analysis of the log-linear fitting coefficients was used and it was determined that the molecular mass estimates of proteins targets are accurate using proximal protein marker electromigration, if the protein markers are released in a microwell that is located≤2.5 mm (center-to-center pitch) from the microwell containing the cell of interest (Equation 1):

$$\hat{γ} = \frac{1}{2 N} {\sum }_{i = 1}^{N} {( {Z}_{i} - {Z}_{i + h} )}^{2} . \qquad ( 1 )$$

No significant difference (Kruskal Wallis test with Dunn's multiple comparisons test, p>0.05) in molecular mass error was observed when protein sizing of endogenous targets was performed either using the protein markers from the same PAGE separation lane or in “proximal” separation lanes (i.e., located≤2.5 mm from the PAGE separation lane containing the proteins targets).

Imidazole Interferes with Protein Photoblotting.

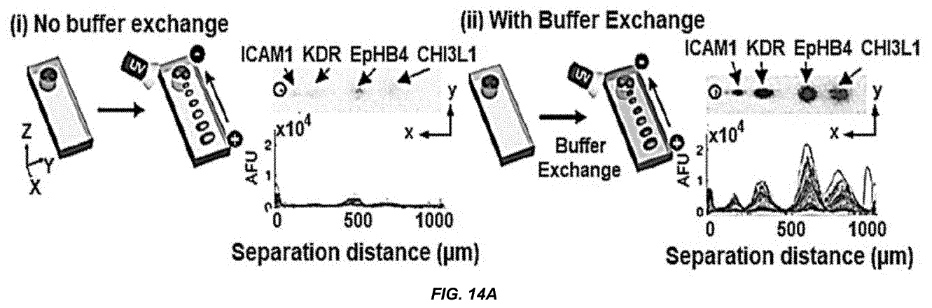

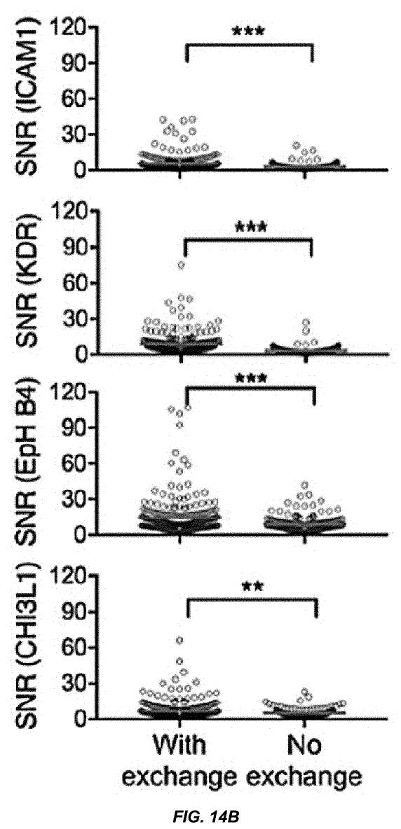

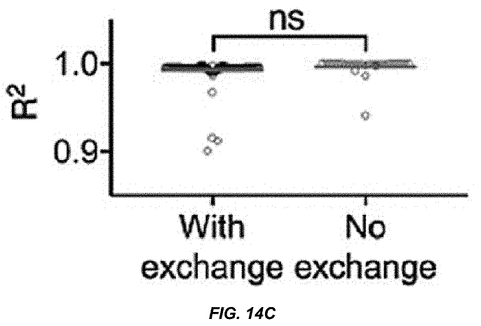

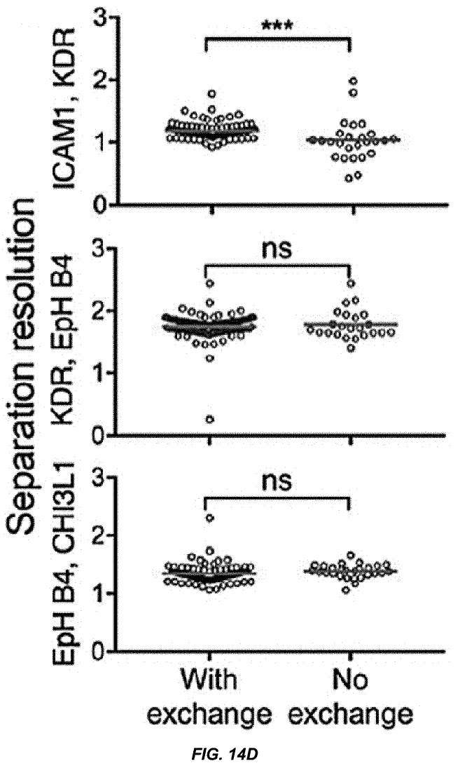

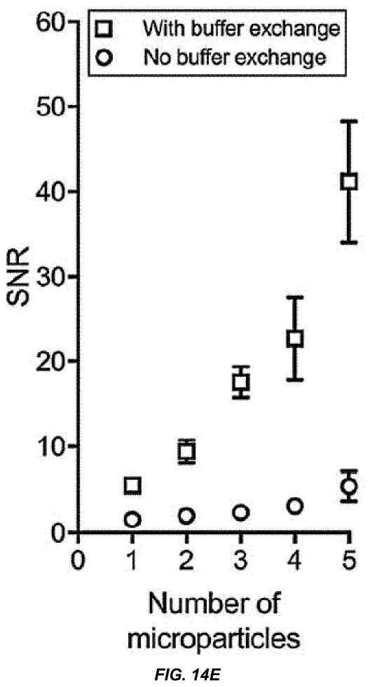

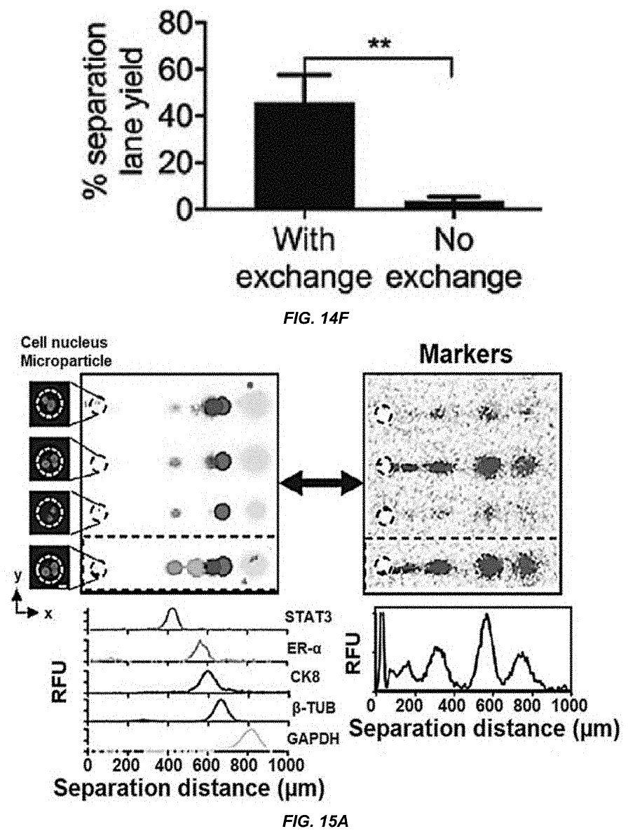

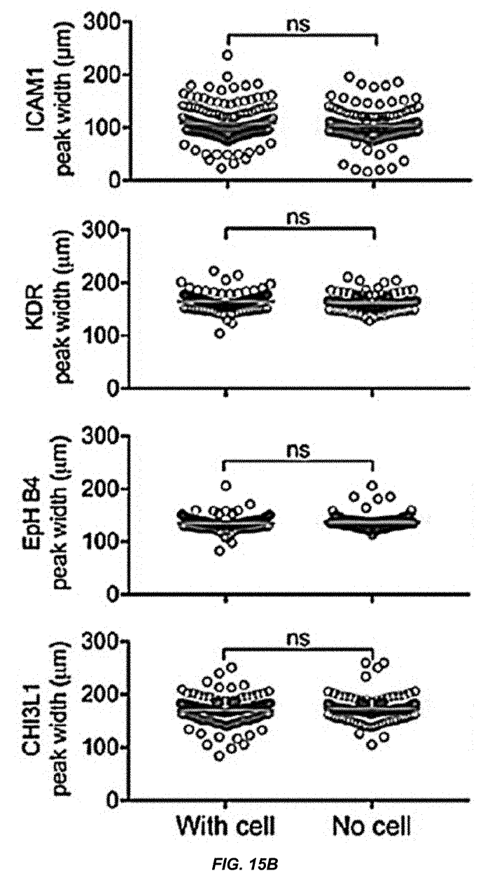

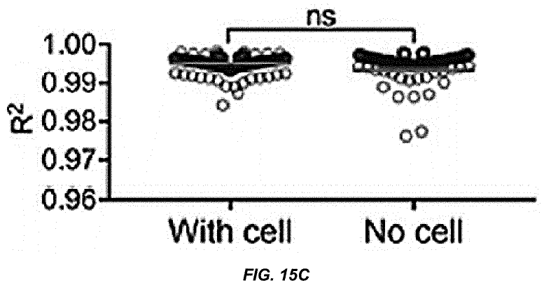

After protein PAGE, efficient photoblotting is critical for protein detection and immunoprobing readout of the single-cell western. During photoblotting. Interference was expected between the imidazole (1 M) and the UV-activated benzophenone group, as benzophone can abstract hydrogen from a secondary amine group on the imidazole, thus allowing benzophenone to form a covalent bond with the high concentration imidazole. In some sense, the imidazole would act to ‘block’ the benzophenone from forming covalent bonds with protein targets. When photoblotting was performed in the presence of 1 M imidazole, just 3.7% of separation lanes with protein marker signals that exceeded the minimum signal to noise ratio (SNR), as described in the quality control protocol was observed (FIG. 14A, 14F).

To enhance the photoblotting efficiency of the protein markers, the standard single-cell western blot workflow was modified to include a buffer-exchange step between the chemical cell lysis step and the protein PAGE step. This buffer exchange step reduces the imidazole concentration in the polyacrylamide gel matrix prior to the UV photoblotting step. A similar approach to buffer exchange has been employed to reduce Joule heating during single-cell electrophoresis. Comparing mean SNR values for the protein markers, the SNR with the buffer exchange step was >20% greater than the SNR without the buffer exchange step (FIG. 14B). For endogenous proteins in each MCF-7 cell lysate, the buffer exchange increased immunoprobing signal from β-tubulin (β-TUB), cytokeratin 8 (CK8), and GAPDH to levels observed with no imidazole included in the chemical lysis step (FIG. 16).

Considering total assay performance, experiments were performed to examine whether the buffer exchange might affect the goodness of fit in the log-linear regression and separation resolution (SR) of the protein markers (FIG. 14C-D, Equation 2).

$${R}_{s} = \frac{Δ x}{\frac{1}{2} ( 4 {σ}_{1} + 4 {σ}_{2} )} \qquad ( 2 )$$

where Δx is the center-to-center distance between the protein peaks and σ is the peak width. A separation resolution greater than one was achieved for each adjacent protein pair in a four-protein standard spanning ˜40-100 kDa.

The goodness of fit distribution of R² values with the buffer exchange was not significantly different from that without the buffer exchange (unpaired t test, p=0.20, n_{with}_{_}_{exchange}=107, nₙₒ_{_}_{exchange}=27; FIG. 14C). Furthermore, no decrease in SR between the protein markers was detectable (unpaired t test; SR_{ICAM1%KDR}, p=0.0003; SR_{KDR&EpH} B4, p=0.55; SR_{EpH B4&CHI3L1}, p=0.19; FIG. 14D). In fact, the median and mean SR between ICAM1 and KDR were slightly greater with the buffer exchange (μ=1.18, median=1.17), as compared conditions without the buffer exchange (p=1.03, median=1.02; FIG. 14D). Joule heating was not reduced upon buffer exchange, nor were corresponding improvements in SR observed.^{[65]} The buffer exchange here utilized room temperature lysis buffer that simply omits imidazole, while still containing high concentrations of conductive ionic detergents that result in Joule heating-induced peak dispersion during electrophoresis.

Detection Threshold: Minimum Number of Microparticles Per Microwell.

Experiments were performed to assess the number of microparticles per microwell required to detect protein markers with an SNR>3. The KDR protein was used to assess the relationship between SNR of protein peaks (E=40 V/cm; Δt=25 s) and the number of microparticles per microwell. As expected, the SNR increased with an increasing number of microparticles per microwell (FIG. 14E). Without buffer exchange, >3 microparticles per microwell were required to achieve an SNR≥3 (FIG. 14E). In contrast, the buffer exchange and dilution of imidazole yielded SNR>5 with a single microparticle in a microwell (FIG. 14E). As compared to just 3.7% of separation lanes yielding suitable detection signal without the buffer exchange, under these conditions 55% of lanes yielding an acceptable signal (FIG. 14F).

In formulating ‘design rules’ for protein sizing markers based on these findings, one microparticle per microwell was sufficient for detection of the protein markers in single-cell western blotting. In cases where no microparticles are loaded into a cell-laden microwell, proximal microwells containing protein markers may be used for cellular protein sizing. In cases where protein targets are >100 kDa, microparticle loading of the cell-laden microwell should not exceed 3 microparticles, as observed non-specific signal proximal to each microwell—that may interfere with detection of large protein markers—when ≥4 microparticles were seated in a single microwell.

Determining Molecular Mass of Endogenous Protein Targets in MCF-7 Cells.

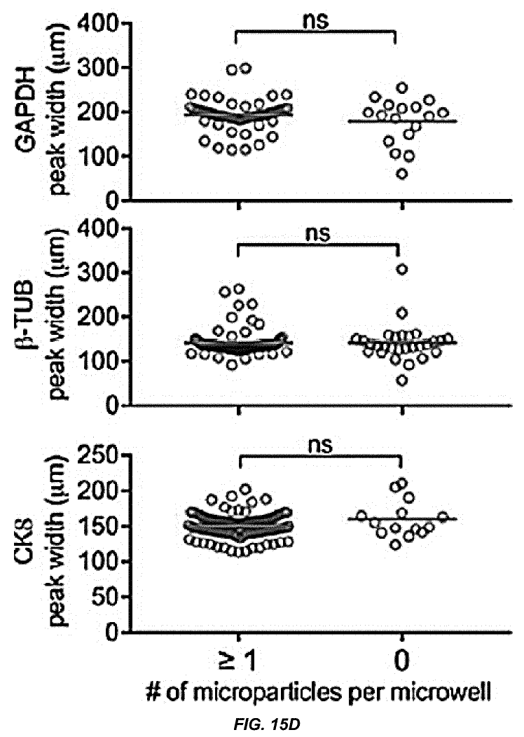

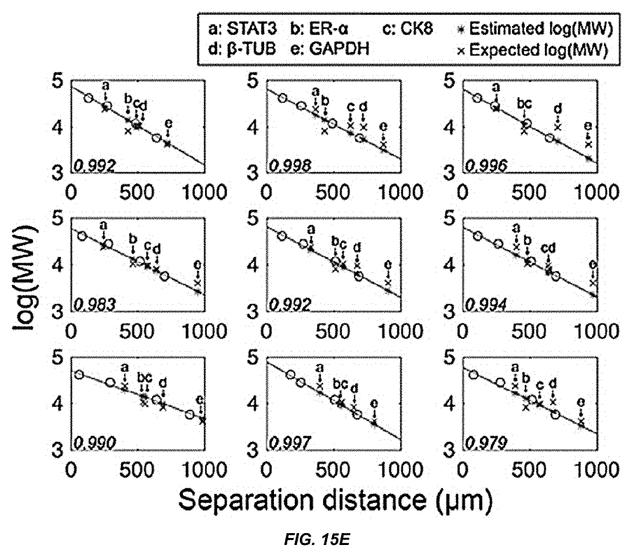

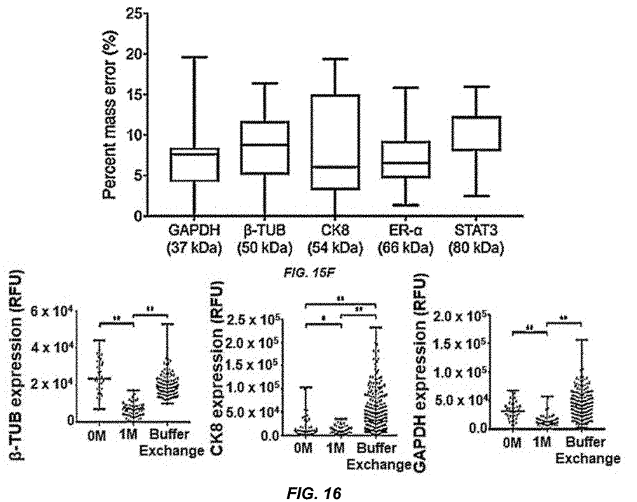

Next, the protein markers was applied to estimate the molecular masses of endogenous proteins from single MCF-7 cells (FIG. 15). For microwells containing both individual MCF-7 breast cancer cells and ≥1 microparticles, single-cell western blotting was performed and concurrently resolved protein markers and endogenous proteins in all PAGE separation lanes (FIG. 15A).

Experiments were performed to identify any confounding interactions when running protein PAGE on the protein marker and single-cell lysate from the same microwell. No significant difference in the distribution of the peak widths of the protein markers in the presence of MCF-7 cells (μ±σ; ICAM1: 108.5±34.5, KDR: 165.6±22.8, EpH B4: 135.8±12.5, CHI3L1: 170.4±24.2), as compared to the same protein marker analysis without cells present (μ±σ; ICAM1: 118.1±27.1, KDR: 160.3±25.8, EpH B4: 137.3±12.1, CHI3L1: 176.5±21.9) was observed. The comparison suggests that negligible interference arises from co-loading, cell lysis concurrent with protein marker solubilization, and concurrent sample injection (FIG. 15B; unpaired t-test and F-test, p>0.05, n_{with}_{_}_{cell}=141, nₙₒ_{_}_{cell}=127). The goodness of the log-linear fit to the protein markers remained R²>0.97 and was not significantly different from conditions in which no cells were loaded into the microwells (FIG. 15C; unpaired t test and F test, p>0.05, n_{with}_{_}_{cell}=141, nₙₒ_{_}_{cell}=127).

Reciprocally, for microwells with and without microparticles, experiments were performed to analyze the immunoprobed endogenous proteins to identify any confounding effects. The endogenous proteins expressed at median or higher copy numbers of the mammalian proteome were analyzed. GAPDH, β-TUB, CK8, ER-α, and STAT3 have copy numbers above the limit of detection for the single-cell western blot (˜27,000 copies of protein immobilized in the gel). Experiments were performed to analyze GAPDH, β-TUB, CK8 and the results showed that the protein peak widths were not notably affected by the presence of protein-loaded microparticles in the same microwells ([median_{≥1 microparticle}, median_{0 microparticle}]; GAPDH [193.4, 192.5], β-TUB [134.3, 136.9], CK8 [151.3, 151.9]; Mann-Whitney test, p>0.05; FIG. 15D).

Next, the protein markers were applied to estimate the molecular masses of endogenous proteins from single MCF-7 cells (FIG. 15). For microwells containing both individual MCF-7 breast cancer cells and ≥1 microparticles, single-cell western blotting was performed with concurrently resolved protein markers and endogenous proteins in all PAGE separation lanes (FIG. 15A).

A log-linear regression equation was developed from the protein markers and the measured peak location for each target (for ladders and single-cell western blots performed within the ≤2.5 mm mentioned earlier). About >100 single-cell western blots with protein markers that passed quality control and a R²≥0.97 for the log-linear regression. In these cases, the observed molecular masses of GAPDH (39.6 kDa), β-TUB (47.0 kDa), CK8 (54.3 kDa), and ER-α (66 kDa) and the reported molecular masses (determined by conventional slab-gel western blots) agreed with both median and mean mass errors of <10% (FIG. 15E-F). Out of all endogenous protein targets, STAT3 had the highest mean (10.3%) and median (12.2%) mass error values (FIG. 15F). Conventional slab-gel western blotting reports three protein peaks within the 70-100 kDa range when probing with the STAT3 antibody, possibly slightly reducing the accuracy of target sizing in the single-cell western blot, which has lower resolving power than the 4 cm-long separation lengths utilized for pooled cell lysate analysis in slab gels. Overall, single-cell western blotting incorporating the protein markers resulted in mean and median mass errors<12% for all endogenous protein targets from the MCF-7 cells (FIG. 15F). This observed performance is comparable to the mass sizing performance of both slab-gel PAGE and microfluidic sizing chips (Agilent Bioanalyzer®), which reports mass errors ˜10%. Not surprisingly, analysis showed lower protein target mass errors estimating mass using the microparticle-delivered protein markers as compared to using endogenous proteins from cell lysate as markers (FIG. 13B, 15F). For example, the mean and median mass errors for CK8 were 40% lower when determined by the microparticle-delivered protein markers, as compared to using endogenous cellular protein targets. The microparticle protein marker vehicle thus find utility in assigning molecular masses to proteoforms, including truncated isoforms with >20% molecular mass differences from the full-length protein (FIG. 15F).

A number of embodiments have been described herein. Nevertheless, it will be understood that various modifications may be made without departing from the spirit and scope of this disclosure. Accordingly, other embodiments are within the scope of the following claims.

What is claimed is:

1. A method for controlling biomolecule measurement quality in a gel-microfluidic device, comprising:
introducing into a microfluidic device one or more nitrilotriacetic acid (NTA)-functionalized microparticles that comprise one or more reversibly attached tracer molecules;
releasing the tracer biomolecules from the microparticles by using a releasing agent in a buffer and/or heating; and
measuring the released tracer biomolecules.

2. The method of claim 1, wherein the NTA-functionalized particles comprising one or more reversibly attached tracer biomolecules are introduced into the microfluidic device by either using passive gravity, magnetic attraction, or centrifugal forces.

3. The method of claim 1 or claim 2, wherein the NTA-functionalized particles are NTA-functionalized magnetic particles.

4. The method of claim 3, wherein the NTA-functionalized magnetic particles comprise Fe₂O₃, Fe₃O₄, Ni²⁺, or Co²⁺.

5. The method of claim 1, wherein the NTA-functionalized particles are NTA-functionalized polystyrene, silica, or polyketal particles.

6. The method of claim 1, wherein the one or more tracer molecules are reversibly attached to the NTA-functionalized particles using chelation click chemistry.

7. The method of claim 1, wherein the one or more tracer molecules are proteins, peptides or ribosomes.

8. The method of claim 1, wherein the one or more tracer molecules are one or more proteins that differ by molecular weight.

9. The method of claim 8, wherein the one or more proteins are fluorescently labeled.

10. The method of claim 1, wherein the one or more tracer molecules comprise polyhistidine tags.

11. The method of claim 8, wherein the one or more proteins comprise 6× histidine tags located at the C′ or N′ terminus of the proteins.

12. The method of claim 1, wherein the releasing agent is a competitive ligand.

13. The method of claim 12, wherein the releasing agent is imidazole.

14. The method of claim 1, wherein the released tracer molecules can be measured by measuring fluorescent light intensity.

15. The method of claim 1, wherein the released tracer molecules can be measured by using an antibody that is linked to a reporter enzyme that is capable of cleaving chemiluminescent agents, and measuring luminescent light intensity.

16. The method of claim 1, wherein the microfluidic device is a single-cell mass cytometry device.

17. The method of claim 16, wherein the microfluidic device is a single-cell mass cytometry device comprising polyacrylamide.

18. A method for using protein-coated microparticles for measurement standardization in single-cell protein assays, comprising:
introducing into one or more wells of a single cell electrophoretic cytometry device:
(i) a cell; and
(ii) one or more nitrilotriacetic acid (NTA)-functionalized particles that comprise one or more reversibly attached proteins;
releasing the one or more proteins from the particles by using a cell lysis buffer which comprises imidazole; and
optionally performing buffer exchange to remove the imidazole;
separating the one or more proteins by applying an electric field;
immobilizing the one or more proteins by using ultra violet light;
quantifying one or more proteins using immunoprobing or by measuring fluorescence or luminescence light intensity.

19. The method of claim 18, wherein the single-cell protein assay is single cell-resolution western blotting.

20. The method of claim 18, wherein the cell and the one or more NTA-functionalized microparticles are loaded into wells of a polyacrylamide gel electrophoresis (PAGE) gel.

21. The method of claim 18, wherein the one or more proteins comprise polyhistidine tags and are reversibly attached to the NTA-functionalized particles using chelation click chemistry.
